(19) United States
(12) Patent Application Publication
(10) Pub. No.: US 2025/0186734 A1
(43) Pub. Date: Jun. 12, 2025

(54) METHODS AND SYSTEMS FOR AUTOMATED NEUROCOGNITIVE TRAINING TO EXTEND RELIEF OF TREATMENT-RESISTANT MENTAL DISORDERS

(71) Applicant: UNIVERSITY OF PITTSBURGH-OF THE COMMONWEALTH SYSTEM OF HIGHER EDUCATION, Pittsburgh, PA (US)

(72) Inventor: Rebecca B. PRICE, Pittsburgh, PA (US)

(21) Appl. No.: 18/842,570

(22) PCT Filed: Mar. 2, 2023
(86) PCT No.: PCT/US2023/014336

§ 371 Date: Aug. 29, 2024

Related U.S. Application Data
(60) Provisional application No. 63/316,098, filed on Mar. 3, 2022.

(51) Int. Cl. A61M 21/00; A61K 31/135; A61K 31/4025; A61K 31/5386; A61K 31/573; A61K 31/58; A61P 25/24

(52) U.S. Cl. CPC A61M 21/00; A61K 31/135; A61K 31/4025; A61K 31/5386; A61K 31/573; A61K 31/58; A61P 25/24; A61M 2021/0027; A61M 2021/005; A61M 2021/0077; A61M 2205/502

(57) ABSTRACT

Methods and systems for automated neurocognitive training to extend relief of treatment-resistant mental disorders are described herein. An example method includes administering a drug having an antidepressant effect to a patient, and administering a neurocognitive training protocol to the patient during a critical time period following administration of the drug. Additionally, the step of administering the neurocognitive training protocol to the patient includes delivering, using a computing device, a plurality of automated neurocognitive training sessions to the patient. A plurality of conditioning sequences are presented to the patient during delivery of each of the automated neurocognitive training sessions, where each respective conditioning sequence includes a respective non-self-relevant stimulus and a respective self-relevant stimulus.

### CROSS-REFERENCE TO RELATED APPLICATIONS

This application claims the benefit of U.S. provisional patent application No. 63/316,098, filed on Mar. 3, 2022, and titled “METHODS AND SYSTEMS FOR AUTOMATED NEUROCOGNITIVE TRAINING TO EXTEND RELIEF OF TREATMENT-RESISTANT MENTAL DISORDERS,” the disclosure of which is expressly incorporated herein by reference in its entirety.

### STATEMENT REGARDING FEDERALLY FUNDED RESEARCH

This invention was made with government support under Grant nos. MH113857 and MH124983 awarded by the National Institutes of Health. The government has certain rights in the invention.

### BACKGROUND

Depression is a leading cause of disability worldwide, with a public disease burden of staggering proportions. While efficacious treatments have been available for decades, remission rates are low, relapse rates are high, and disorder prevalence rates remain notably consistent, with only 12.7% of patients receiving minimally adequate treatment. Suicide, the most tragic outcome of depression, claims 1 million lives globally each year. Suicide rates in the United States have continued to climb in recent years in spite of concerted research and clinical efforts to address this tragic need. 80% of suicide decedents were seen in a health care setting within a year of their death, suggesting the system as a whole is missing critical opportunities to engage these patients in an effective suicide prevention strategy. Novel and innovative treatments that are accessible, portable, and dissemination-friendly, are urgently needed to address these public health crises.

Depression intervention development has increasingly sought to take advantage of technology to increase patient access, reduce cost, and minimize aversive consequences through the use of automated, computer-based procedures/software. The past decade has seen growing interest in automated “cognitive training” (CT) treatments, designed to modulate affective processing patterns directly in order to reduce symptoms; but clinical effects, though showing potential, are unreliable, particularly in acutely depressed patients, and typically take weeks to provide relief. In tandem, rapidly-acting pharmacological agents, such as subanesthetic ketamine, have offered the promise of a breakthrough in the way that depression is managed, but this promise also remains unfulfilled to date. Studies document 55-80% response rates in depressed patients as early as 2 hours post-ketamine, even among patients resistant to conventional treatments, suggesting the potential to bring immediate relief to many. Yet, effects of a single infusion dissipate almost as rapidly (3-7 days), raising significant challenges for clinical translation in light of concerns regarding the safety and feasibility of repeated ketamine. Furthermore, the majority of depressed patients express preference for treatments involving a behavioral/learning component (e.g., up to 91% enrolling in drug trials). Practical challenges in maintaining drug regimens over time further suggest that a diversity of approaches to achieve rapid, enduring relief is needed.

### SUMMARY

In some implementations, the techniques described herein relate to a method for treating a patient having a mental disorder including: administering a drug having an antidepressant effect to the patient; and administering a neurocognitive training protocol to the patient during a critical time period following administration of the drug. The step of administering the neurocognitive training protocol to the patient includes delivering, using a computing device, a plurality of automated neurocognitive training sessions to the patient, wherein a plurality of conditioning sequences are presented to the patient during delivery of each of the automated neurocognitive training sessions, each respective conditioning sequence including a respective non-self-relevant stimulus and a respective self-relevant stimulus.

In some implementations, a first automated neurocognitive training session and a second automated neurocognitive training session are delivered to the patient on a first day, and a third automated neurocognitive training session and a fourth automated neurocognitive training session are delivered to the patient on a second day. Optionally, a period of time between delivery of the first and second automated neurocognitive training sessions to the patient is about 20 minutes or more. Optionally, a period of time between delivery of the third and fourth automated neurocognitive training sessions to the patient is about 20 minutes or more.

In some implementations, each of the automated neurocognitive training sessions has a length between about 15 minutes and about 20 minutes.

In some implementations, the plurality of automated neurocognitive training sessions is between about 2 sessions and about 14 sessions. Optionally, the plurality of automated neurocognitive training sessions is about 8 sessions.

In some implementations, each respective conditioning sequence further includes a task, the task being presented to the patient following or in relation to presentation of a respective non-self-relevant and self-relevant stimuli pair.

In some implementations, the respective non-self-relevant stimulus and/or the respective self-relevant stimulus for a respective conditioning sequence is an auditory stimulus. In some implementations, the respective non-self-relevant stimulus and/or the respective self-relevant stimulus for a respective conditioning sequence is a visual stimulus.

In some implementations, the respective self-relevant stimulus for a respective conditioning sequence includes a sound, image, or word that is associated with the patient. Optionally, the respective non-self-relevant stimulus for the respective conditioning sequence includes a sound, image, or word that is associated with a positive meaning. Optionally, the respective non-self-relevant stimulus for the respective conditioning sequence includes a sound, image, or word that is associated with a life-affirming meaning.

In some implementations, the respective non-self-relevant stimulus and the respective self-relevant stimulus for a respective conditioning sequence are included in an n×m matrix including a plurality of visual stimuli. Optionally, the step of administering the neurocognitive training protocol to the patient further includes receiving, from the patient, a selection matching the respective non-self-relevant stimulus and the respective self-relevant stimulus for the respective conditioning sequence.

In some implementations, one or more of the plurality of conditioning sequences includes a subliminal presentation of a non-self-relevant stimulus or a self-relevant stimulus. In some implementations, one or more of the plurality of conditioning sequences includes a supraliminal presentation of a non-self-relevant stimulus or a self-relevant stimulus.

In some implementations, the computing device includes a processor and a memory operably coupled to the processor, the memory having computer-executable instructions stored thereon. Optionally, the memory has computer-executable instructions stored thereon, that when executed by the processor, cause the processor to generate each respective conditioning sequence.

In some implementations, the computing device further includes a display device operably coupled to the processor, and wherein the memory has computer-executable instructions stored thereon, that when executed by the processor, cause the processor to: generate graphical data for each respective conditioning sequence; and cause the graphical data to be displayed on the display device.

In some implementations, the critical time period is a period during which the drug acutely proliferates synaptic contacts within the patient's brain. In some implementations, the critical time period is between about 2 hours and about 14 days following administration of the drug. In some implementations, the critical time period is between about 1 day and about 5 days following administration of the drug.

In some implementations, the drug is a rapid-acting antidepressant. In some implementations, the rapid-acting antidepressant is ketamine. In some implementations, the drug is brexanolone, zuranolone, rapastinel, scopolamine, or a psychedelic.

In some implementations, the mental disorder is depression. In some implementations, the mental disorder is suicidal behavior.

In some implementations, the techniques described herein relate to a computer-implemented method for administering a neurocognitive training protocol to a patient having a mental disorder, the patient having received a drug having an antidepressant effect, and the neurocognitive training protocol being administered during a critical time period following administration of the drug to the patient. The computer-implemented method includes: delivering a plurality of automated neurocognitive training session to the patient, wherein a plurality of conditioning sequences are presented to the patient during delivery of each of the automated neurocognitive training sessions, each respective conditioning sequence including a respective non-self-relevant stimulus and a respective self-relevant stimulus.

In some implementations, a first automated neurocognitive training session and a second automated neurocognitive training session are delivered to the patient on a first day, and a third automated neurocognitive training session and a fourth automated neurocognitive training session are delivered to the patient on a second day. Optionally, a period of time between delivery of the first and second automated neurocognitive training sessions to the patient is about 20 minutes or more. Optionally, a period of time between delivery of the third and fourth automated neurocognitive training sessions to the patient is about 20 minutes or more.

In some implementations, each of the automated neurocognitive training sessions has a length between about 15 minutes and about 20 minutes.

In some implementations, the plurality of automated neurocognitive training sessions is between about 2 sessions and about 14 sessions. Optionally, the plurality of automated neurocognitive training sessions is about 8 sessions.

In some implementations, each respective conditioning sequence further includes a task, the task being presented to the patient following or in relation to presentation of a respective non-self-relevant and self-relevant stimuli pair.

In some implementations, the respective non-self-relevant stimulus and/or the respective self-relevant stimulus for a respective conditioning sequence is an auditory stimulus. In some implementations, the respective non-self-relevant stimulus and/or the respective self-relevant stimulus for a respective conditioning sequence is a visual stimulus.

In some implementations, the respective self-relevant stimulus for a respective conditioning sequence includes a sound, image, or word that is associated with the patient. Optionally, the respective non-self-relevant stimulus for the respective conditioning sequence includes a sound, image, or word that is associated with a positive meaning. Optionally, the respective non-self-relevant stimulus for the respective conditioning sequence includes a sound, image, or word that is associated with a life-affirming meaning.

In some implementations, the respective non-self-relevant stimulus and the respective self-relevant stimulus for a respective conditioning sequence are included in an n×m matrix including a plurality of visual stimuli. Optionally, the computer-implemented method further includes receiving, from the patient, a selection matching the respective non-self-relevant stimulus and the respective self-relevant stimulus for the respective conditioning sequence.

In some implementations, one or more of the plurality of conditioning sequences includes a subliminal presentation of a non-self-relevant stimulus or a self-relevant stimulus. In some implementations, one or more of the plurality of conditioning sequences includes a supraliminal presentation of a non-self-relevant stimulus or a self-relevant stimulus.

In some implementations, the computer-implemented method further includes generating each respective conditioning sequence.

In some implementations, the computer-implemented method further includes generating graphical data for each respective conditioning sequence; and causing the graphical data to be displayed on a display device.

In some implementations, the critical time period is a period during which the drug acutely proliferates synaptic contacts within the patient's brain. In some implementations, the critical time period is between about 2 hours and about 14 days following administration of the drug. In some implementations, the critical time period is between about 1 day and about 5 days following administration of the drug.

In some implementations, the techniques described herein relate to a system for administering a neurocognitive training protocol to a patient having a mental disorder, the patient having received a drug having an antidepressant effect, and the neurocognitive training protocol being administered during a critical time period following administration of the drug to the patient. The system includes: a processor and a memory operably coupled to the processor, the memory having computer-executable instructions stored thereon that, when executed by the processor, cause the processor to: deliver a plurality of automated neurocognitive training session to the patient, wherein a plurality of conditioning sequences are presented to the patient during delivery of each of the automated neurocognitive training sessions, each respective conditioning sequence including a respective non-self-relevant stimulus and a respective self-relevant stimulus.

In some implementations, a first automated neurocognitive training session and a second automated neurocognitive training session are delivered to the patient on a first day, and a third automated neurocognitive training session and a fourth automated neurocognitive training session are delivered to the patient on a second day. Optionally, a period of time between delivery of the first and second automated neurocognitive training sessions to the patient is about 20 minutes or more. Optionally, a period of time between delivery of the third and fourth automated neurocognitive training sessions to the patient is about 20 minutes or more.

In some implementations, each of the automated neurocognitive training sessions has a length between about 15 minutes and about 20 minutes.

In some implementations, the plurality of automated neurocognitive training sessions is between about 2 sessions and about 14 sessions. Optionally, the plurality of automated neurocognitive training sessions is about 8 sessions.

In some implementations, each respective conditioning sequence further includes a task, the task being presented to the patient following or in relation to presentation of a respective non-self-relevant and self-relevant stimuli pair.

In some implementations, the respective non-self-relevant stimulus and/or the respective self-relevant stimulus for a respective conditioning sequence is an auditory stimulus. In some implementations, the respective non-self-relevant stimulus and/or the respective self-relevant stimulus for a respective conditioning sequence is a visual stimulus.

In some implementations, the respective self-relevant stimulus for a respective conditioning sequence includes a sound, image, or word that is associated with the patient. Optionally, the respective non-self-relevant stimulus for the respective conditioning sequence includes a sound, image, or word that is associated with a positive meaning. Optionally, the respective non-self-relevant stimulus for the respective conditioning sequence includes a sound, image, or word that is associated with a life-affirming meaning.

In some implementations, the respective non-self-relevant stimulus and the respective self-relevant stimulus for a respective conditioning sequence are included in an n×m matrix including a plurality of visual stimuli.

In some implementations, one or more of the plurality of conditioning sequences includes a subliminal presentation of a non-self-relevant stimulus or a self-relevant stimulus. In some implementations, one or more of the plurality of conditioning sequences includes a supraliminal presentation of a non-self-relevant stimulus or a self-relevant stimulus.

In some implementations, the memory has further computer-executable instructions stored thereon that, when executed by the processor, cause the processor to generate each respective conditioning sequence.

In some implementations, the memory has further computer-executable instructions stored thereon that, when executed by the processor, cause the processor to generate graphical data for each respective conditioning sequence; and cause the graphical data to be displayed on a display device.

In some implementations, the techniques described herein relate to a method for treating a patient having a mental disorder including: administering a drug having an antidepressant effect to the patient; and administering a neurocognitive training protocol to the patient during a critical time period following administration of the drug. The step of administering the neurocognitive training protocol to the patient includes delivering, using a computing device, a plurality of automated neurocognitive training sessions to the patient. Additionally, each respective automated neurocognitive training session includes: presenting, using the computing device, an n×m matrix including a plurality of visual stimuli to the patient, the plurality of visual stimuli including a first matching pair of non-self-relevant and self-relevant visual stimuli; and receiving, using the computing device, a selection by the patient of the first matching pair of non-self-relevant and self-relevant visual stimuli.

In some implementations, each respective automated neurocognitive training session further includes: presenting, using the computing device, a second matching pair of non-self-relevant and self-relevant visual stimuli to the patient; and presenting, using the computing device, a task to the patient following or in relation to presentation of the second matching pair of non-self-relevant and self-relevant visual stimuli.

In some implementations, each respective automated neurocognitive training session further includes: presenting, using the computing device, a self-relevant visual stimulus to the patient; presenting, using the computing device, a task to the patient following or in relation to presentation of the self-relevant stimulus; and presenting, using the computing device, a positive non-self-relevant visual stimulus following successful completion of the task by the patient.

In some implementations, each of the automated neurocognitive training sessions has a length between about 15 minutes and about 20 minutes.

In some implementations, the critical time period is a period during which the drug acutely proliferates synaptic contacts within the patient's brain. In some implementations, the critical time period is between about 2 hours and about 14 days following administration of the drug. In some implementations, the critical time period is between about 1 day and about 5 days following administration of the drug.

In some implementations, the techniques described herein relate to a computer-implemented method for administering a neurocognitive training protocol to a patient having a mental disorder, the patient having received a drug having an antidepressant effect, and the neurocognitive training protocol being administered during a critical time period following administration of the drug to the patient. The computer-implemented method includes: presenting an n×m matrix including a plurality of visual stimuli to the patient, the plurality of visual stimuli including a first matching pair of non-self-relevant and self-relevant visual stimuli; and receiving a selection by the patient of the first matching pair of non-self-relevant and self-relevant visual stimuli.

In some implementations, the techniques described herein relate to a system for administering a neurocognitive training protocol to a patient having a mental disorder, the patient having received a drug having an antidepressant effect, and the neurocognitive training protocol being administered during a critical time period following administration of the drug to the patient. The system includes: a processor and a memory operably coupled to the processor, the memory having computer-executable instructions stored thereon that, when executed by the processor, cause the processor to: present an n×m matrix including a plurality of visual stimuli to the patient, the plurality of visual stimuli including a first matching pair of non-self-relevant and self-relevant visual stimuli; and receive a selection by the patient of the first matching pair of non-self-relevant and self-relevant visual stimuli.

In some implementations, the techniques described herein relate to a method for treating a patient having a mental disorder including: administering a drug having an antidepressant effect to the patient; and administering a neurocognitive training protocol to the patient during a critical time period following administration of the drug. The step of administering the neurocognitive training protocol to the patient includes delivering, using a computing device, a plurality of automated neurocognitive training sessions to the patient, wherein a plurality of conditioning sequences are presented to the patient during delivery of each of the automated neurocognitive training sessions.

In some implementations, the techniques described herein relate to a method for conditioning a therapeutic association in a patient including: administering a neuroplasticity-enhancing treatment to the patient; and administering a neurocognitive training protocol to the patient during a critical time period following administration of the neuroplasticity-enhancing treatment. The step of administering the neurocognitive training protocol to the patient includes delivering, using a computing device, one or more conditioning sequences configured to condition a therapeutic association to the patient. In some implementations, the neuroplasticity-enhancing treatment is a drug. In other implementations, the neuroplasticity-enhancing treatment is a neuromodulatory intervention.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a flowchart illustrating example operations for treating a subject having a mental disorder according to implementations described herein.

FIG. 2 illustrates an example conditioning sequence delivered during neurocognitive training according to implementations described herein.

FIG. 3 illustrates another example conditioning sequence delivered during neurocognitive training according to implementations described herein.

FIG. 4 illustrates another example conditioning sequence delivered during neurocognitive training according to implementations described herein.

FIG. 5 illustrates an example incidental task (i.e., lexical decision) delivered during neurocognitive training according to implementations described herein.

FIG. 6 illustrates another example incidental task (i.e., rapid selection) delivered during neurocognitive training according to implementations described herein

FIGS. 7A-7C illustrates another example conditioning sequence delivered during neurocognitive training according to implementations described herein. FIG. 7A illustrates an example user interface. FIG. 7B illustrates example self-relevant and non-self relevant stimuli pairs.

FIG. 7C illustrates an example matrix.

FIG. 8 is an example computing device.

FIG. 9 is a chart illustrating depression severity scores (total Montgomery-Asberg Depression Rating scale scores) as a function of days since infusion and treatment allocation in a randomized controlled trial of ketamine and automated self-association training (ASAT) [ketamine+active ASAT, n=53; ketamine+sham ASAT, n=50; saline+active ASAT, n=51]. Line plots represent mean values and error bars represent standard error of the mean within each timepoint and treatment group.

FIG. 10 is Table S1 illustrating patient and clinician guesses regarding treatment allocation, as a function of true allocations.

FIG. 11A-11D are Table S2 illustrating adverse events. FIG. 11A is for ketamine-treated patients, adverse events reported on infusion day. FIG. 11B is for ketamine-treated patients, adverse events reported after infusion day. FIG. 11C is for saline-treated patients, adverse events reported on infusion day. FIG. 11D is for saline-treated patients, adverse events reported after infusion day.

FIG. 12 is Table S3 illustrating robustness of primary findings in main text when including covariates in models.

FIG. 13 illustrates depression severity scores (total Quick Inventory of Depressive Symptoms: Self-Report scale scores) as a function of days since infusion and treatment allocation according to an example described herein.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” an, “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for using automated neurocognitive training following administration of intravenous ketamine to extend relief of treatment-resistant depression, it will become evident to those skilled in the art that the implementations are not limited thereto, but are applicable for using automated neurocognitive training following administration of other drugs, compounds, or agents to extend relief of treatment-resistant depression or other mental disorders.

As used herein, the terms “about” or “approximately” when referring to a measurable value such as an amount, a percentage, and the like, is meant to encompass variations of ±20%, ±10%, ±5%, or ±1% from the measurable value.

“Administration” of “administering” to a subject includes any route of introducing or delivering to a subject an agent. Administration can be carried out by any suitable means for delivering the agent. Administration includes self-administration and the administration by another.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

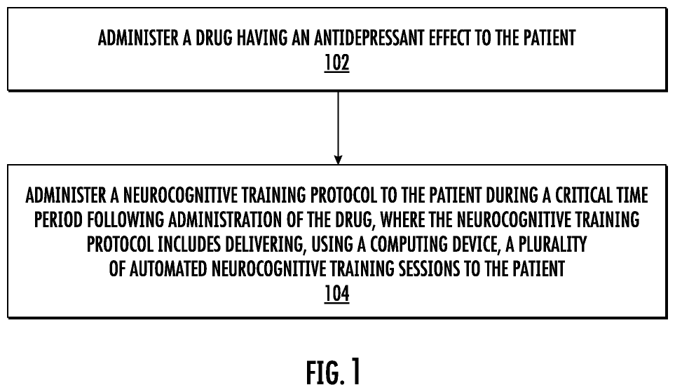

Referring now to FIG. 1, a flowchart illustrating example operations for treating a subject (also referred to herein as a “patient”) having a mental disorder is shown. The methods described herein have advantages over conventional methods including, but not limited to, overcoming the clinical unreliability of cognitive training in patients having depression and/or overcoming the rapid dissipation of the effects of ketamine after a single administration. Other advantages of the methods described herein over the current standard-of-care for mental conditions (such as depression) include that they: 1) have a rapid onset of beneficial effect (e.g., within 1 day); 2) are efficient, requiring minimal investment of time from patients; 3) have an enduring effect of at least 3 months following a brief course of treatment; 4) are fully automated and portable (except for the drug administration), enabling them to be scalable and readily rolled out in a wide variety of clinical and non-clinical settings; 5) are not resource-intensive to administer, requiring minimal investment of time from clinicians and medical professionals; and 6) are efficacious even in treatment-resistant patients who previously did not benefit from standard treatments. The methods described herein achieve these advantages by pairing delivery of an antidepressant drug with delivery of automated neurocognitive training according to protocols described herein. Such automated neurocognitive training leverages Pavlovian conditioning in order to promote positive implicit self-representations and self-worth. Additionally, the automated neurocognitive training may include tasks to enhance patient engagement with the training. In some implementations described herein, the mental disorder is depression. Optionally, the mental disorder is treatment-resistant depression, i.e., the patient failed to respond to treatments such as antidepressants and/or psychotherapy. In other implementations described herein, the mental disorder is suicidal behavior. It should be understood that depression and suicidal behavior are provided only as example mental disorders. This disclosure contemplates that the mental disorder may be conditions other than depression and suicidal behavior, including other treatment-resistant mental disorders.

At step 102, a drug having an antidepressant effect is administered to the patient. As discussed above, the patient has a mental disorder, for example treatment-resistant depression. In some implementations, the drug is a rapid-acting antidepressant. As used herein, a rapid-acting antidepressant has a therapeutic onset (e.g., separation observed between the active drug and a control condition in a randomized controlled trial) in a subject between about one minute and three weeks after administration of the drug. This is as opposed to conventional (e.g., the vast majority of Food and Drug Administration (FDA) approved) antidepressants, which typically take between four to eight weeks to produce a therapeutic effect in the subject. Ketamine is an example rapid-acting antidepressant. Ketamine is a well-known medication, commonly used as an anesthetic. Intravenous ketamine has been administered to human subjects and shown promise in reducing symptoms of depression and/or reducing suicidality. It should be understood that ketamine is provided only as an example rapid-acting anti-depressant. This disclosure contemplates administering other rapid-acting antidepressants to the patient including, but not limited to, scopolamine and other NMDA receptor antagonists (see e.g., Witkin J M, Martin A E, Golani L K, Xu N Z, Smith J L. Rapid-acting antidepressants. Adv Pharmacol. 2019; 86:47-96. Doi: 10.1016/bs.apha.2019.03.002. Epub 2019 Apr. 24. PMID: 31378256), brexanolone, or rapastinel. This disclosure also contemplates administering other drugs having antidepressant effect to the patient including, but not limited to, zuranolone or a psychedelic (e.g., psilocybin, lysergic acid diethylamide, ayahuasca, 3,4-Methylenedioxymethamphetamine (MDMA)).

At step 104, a neurocognitive training protocol is administered to the patient during a critical time period following administration of the drug. As used herein, the critical time period is a period during which the drug is expected to acutely proliferate synaptic contacts within the patient's brain. For example, the critical time period can optionally be between about 2 hours and about 14 days following administration of the drug. Optionally, the critical time period is between about 1 day and about 5 days following administration of the drug. It should be understood that the specific critical time periods described above are provided only as examples and may vary depending on the drug administered to the patient.

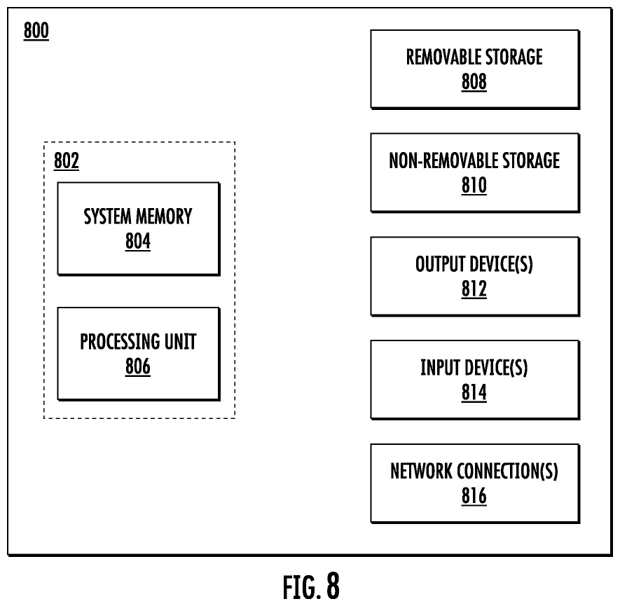

As described herein, the step of administering the neurocognitive training protocol to the patient includes delivering, using a computing device, a plurality of automated neurocognitive training sessions to the patient. This disclosure contemplates that the computing device can be the computing device shown in FIG. 8. Additionally, the computing device can be configured to generate content (e.g., including graphical display data) for the automated neurocognitive training sessions. For example, the computing device can be configured to generate content, which may include audio and/or visual content, for conditioning sequences (discussed below). Optionally, the computing device further includes a display device, and the computing device is further configured to generate graphical data for each conditioning sequence, and cause the graphical data to be displayed on the display device.

As described herein, the neurocognitive training protocol includes delivering a plurality of automated neurocognitive training sessions to the patient. The number of sessions can be between about 2 sessions and about 14 sessions, and optionally about 8 sessions. For example, in one implementation described in the Examples below, eight automated neurocognitive training sessions are delivered to the patient. The eight sessions are delivered twice daily over a period of four consecutive days. Delivery of eight sessions of automated neurocognitive training sessions twice daily over four consecutive days during the critical time period following drug administration was found to be effective in treating depression. It should be understood that the number of sessions, number of sessions per day, and number of days are provided only as an example. This disclosure contemplates delivering different numbers of automated neurocognitive training sessions to achieve therapeutic effect in the patient. This includes varying the number of sessions per days and/or delivery over different numbers of consecutive or nonconsecutive days.

In one example implementation, the automated neurocognitive training sessions include a first automated neurocognitive training session and a second automated neurocognitive training session delivered to the patient on a first day, and a third automated neurocognitive training session and a fourth automated neurocognitive training session delivered to the patient on a second day. The first and second day are different days and optionally consecutive days or nonconsecutive days. Additionally, this disclosure contemplates delivering additional sessions to the patient on the first day and/or the second day, or optionally on a subsequent day such as a third day, a fourth day, a fifth day, etc. Optionally, a period of time between delivery of automated neurocognitive training sessions on the same day is about 20 minutes or more. The lapse in time between sessions (e.g., 20 min) is designed to facilitate ‘spaced learning’ which allows for some brief forms of memory consolidation to occur in between the sessions and is thought, based on the neuroscience of learning, to facilitate retention of information. It should be understood that the 20 minute lapse in time between sessions is provided only as an example. This disclosure contemplates providing any length of time between sessions that facilitates ‘spaced learning’ as described above. Additionally, each of the automated neurocognitive training sessions has a length of less than about 20 minutes, for example between about 15 minutes and about 20 minutes. A session length of 15-20 minutes has been found to be efficient, effective, avoid boredom, and fatigue according to feedback. It should be understood that a session length of 15-20 minutes is provided only as an example. This disclosure contemplates using any session length that facilitates effectiveness while achieving the objectives above.

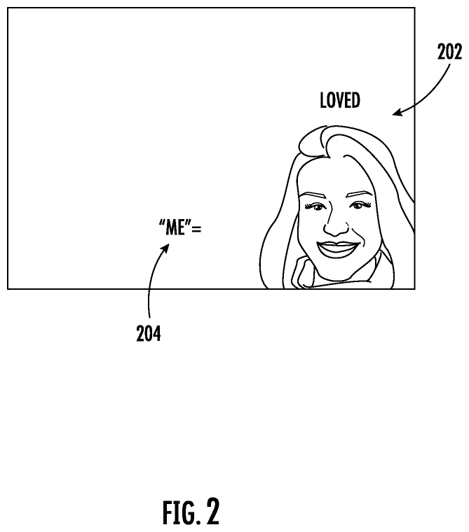

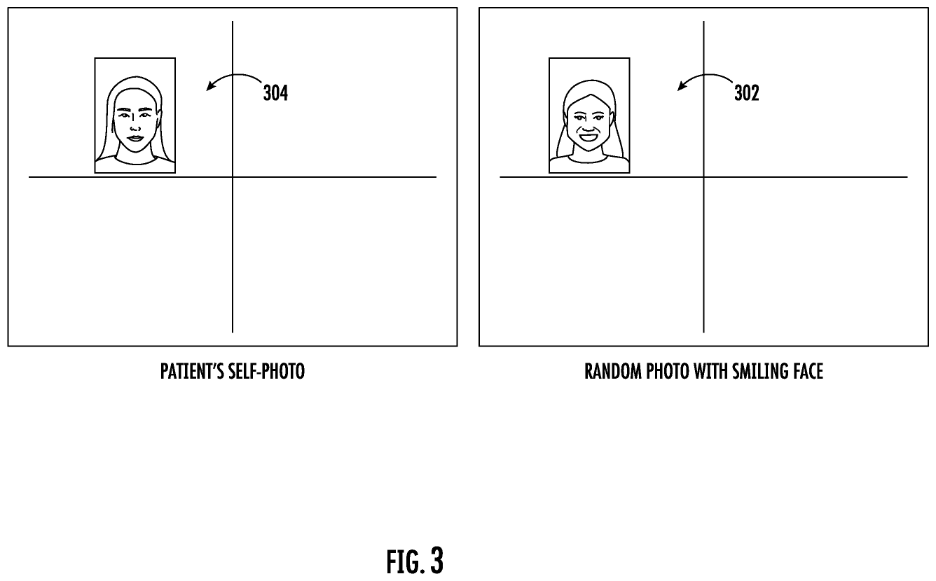

During automated neurocognitive training, a plurality of conditioning sequences are presented to the patient during delivery of each of the automated neurocognitive training sessions. Optionally, in some implementations described herein, each respective conditioning sequence includes a respective non-self-relevant stimulus and a respective self-relevant stimulus. Other conditioning sequences may include, but are not limited to, alternative therapeutic pairings targeting a variety of mental health concerns, such as pairings of a stimulus where approaching the stimulus or activity is desired [e.g., feared (phobic) stimulus; healthy and/or valued activities or scenarios that are presently avoided; cues related to a future-oriented mindset] and a respective positive stimulus; pairings of a respective undesirable stimulus where avoiding the stimulus or activity is desired (e.g., addiction-related craving cue; self-injury-related cue) and a respective negative stimulus. An example conditioning sequence is shown in FIG. 2. The conditioning sequence of FIG. 2 includes a non-self-relevant stimulus 202 (or “unconditioned stimulus (US)”), which is associated with a positive trait (i.e., “LOVED” and the photo of a smiling actor in FIG. 2) such as “sweet,” “attractive,” photos of smiling actors, etc., and a self-relevant stimulus 204 (or “conditioned stimulus (CS)”), which is associated with the patient (i.e., “ME” in FIG. 2) such as “I,” “me,” photo of the patient, etc. It should be understood that the equal sign shown in FIG. 2 is provided only to illustrate the concept of strengthening an association in the patient's brain between the non-self-relevant stimulus 202 and self-relevant stimulus 204. Another example conditioning sequence is shown in FIG. 3. The conditioning sequence of FIG. 3 includes a non-self-relevant stimulus 302, which is a photo of a random smiling person (i.e., a positive trait), and a self-relevant stimulus 304, which is a photo of the subject. Such conditioning sequences are designed to leverage Pavlovian conditioning in order to promote positive implicit self-representations and self-worth. It should be understood that the stimuli shown and described with regard to FIGS. 2 and 3 are only provided as examples. For example, this disclosure contemplates that, in some implementations, the non-self-relevant stimulus and/or the self-relevant stimulus is an auditory stimulus. Alternatively or additionally, in some implementations, the non-self-relevant stimulus and/or the self-relevant stimulus is a visual stimulus. Optionally, the self-relevant stimulus for each respective conditioning sequence includes a sound, image, or word that is associated with the patient. Optionally, the sound image, or word may have a positive meaning, or the sound image, or word may have a life-affirming meaning.

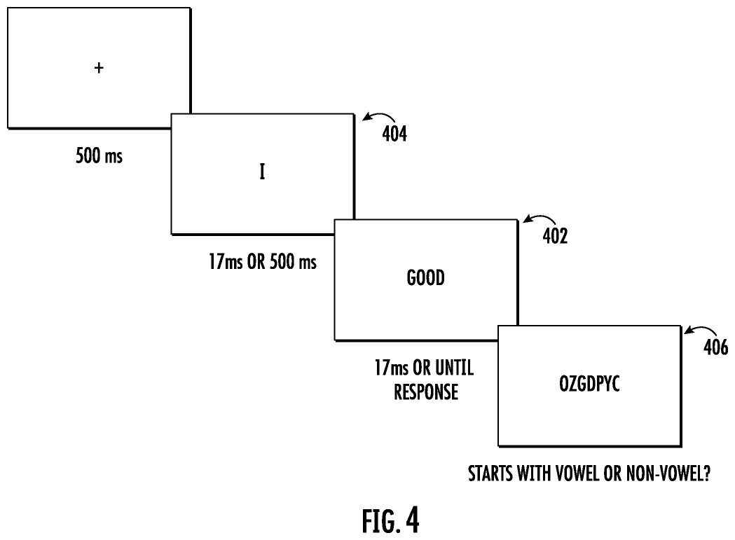

Optionally, in some implementations, one or more of the plurality of conditioning sequences includes a subliminal presentation of a non-self-relevant stimulus or a self-relevant stimulus. Alternatively or additionally, in some implementations, one or more of the plurality of conditioning sequences includes a supraliminal presentation of a non-self-relevant stimulus or a self-relevant stimulus. This is shown in FIG. 4, where a non-self-relevant stimulus 402 (i.e., the word “good”) and a self-relevant stimulus 404 (i.e., the word “I”) are presented in sequence. For example, in some implementations, the non-self-relevant stimulus 402 and the self-relevant stimulus 404 are presented in sequence subliminally. A subliminal stimulus is below the patient's threshold for conscious perception (e.g., 17 millisecond (ms)). In other implementations, the non-self-relevant stimulus 402 and the self-relevant stimulus 404 are presented in sequence supraliminally. A supraliminal stimulus is above the patient's threshold for conscious perception (e.g., 500 ms presentation). It should be understood that the respective durations for subliminal and supraliminal presentation shown in FIG. 4 are only provided as examples. This disclosure contemplates that the respective durations can be of any length below (i.e., subliminal) or above (i.e., supraliminal) conscious threshold, for example, subliminal (e.g., about 12 ms stimulus) or supraliminal (e.g., >250 ms stimulus).

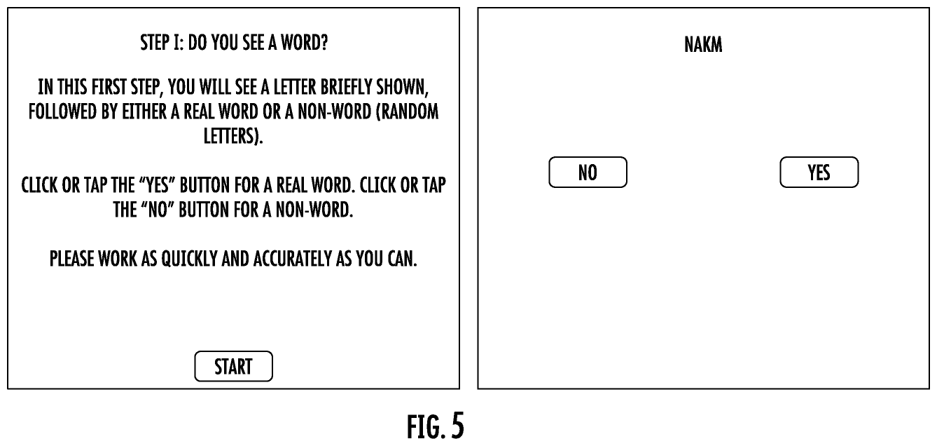

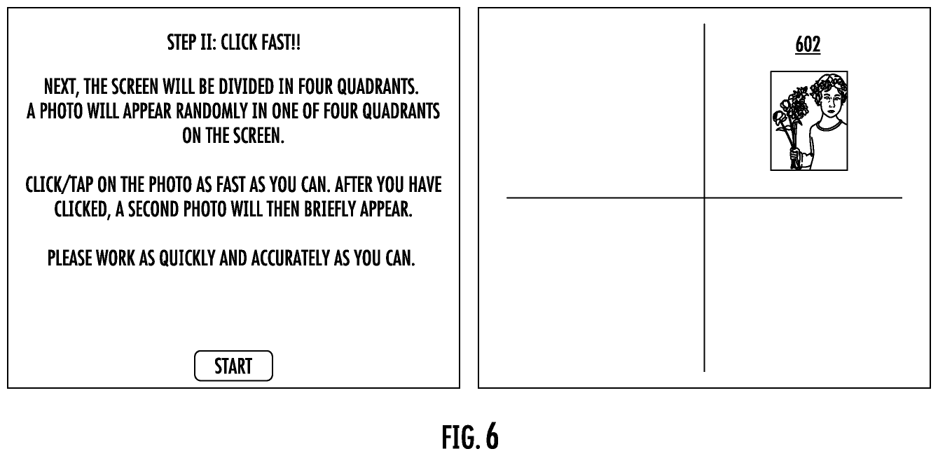

Alternatively or additionally, in some implementations, each respective conditioning sequence further comprises a task, where the task is presented to the patient either following, or in relation to, the presentation of a respective non-self-relevant and self-relevant stimuli pair. This is shown in FIG. 4, where the patient is prompted to respond whether a random stimulus 406 (i.e., the string “ozgdpyc”) starts with a vowel or non-vowel. The task presentation follows presentation of the stimuli pair (i.e., the non-self-relevant stimulus 402 and the self-relevant stimulus 404 pair). In another implementation, the task, a lexical decision task, begins at the point of presentation of the non-self-relevant stimulus (e.g., non-self-relevant stimulus 402 in FIG. 4), and the subject makes a response to the non-self-relevant stimulus to indicate whether the non-self-relevant stimulus comprises a real word or a non-word (e.g., random letter string). Such tasks enhance the patient's engagement with the automatic neurocognitive training, which promotes both semantic and visual processing of the stimuli (e.g., the conditioning sequences that are designed to leverage Pavlovian conditioning in order to promote positive implicit self-representations and self-worth). It should be understood that the task described with respect to FIG. 4 is provided only as an example. This disclosure contemplates presenting another incidental task including, but not limited to, lexical decision tasks or rapid mouse clicking tasks. Another example lexical decision task is illustrated in FIG. 5, where the patient is prompted to label words and non-words (i.e., random character strings), with the words in this task being the non-self-relevant stimuli involved in the conditioning sequence. An example mouse clicking task is illustrated in FIG. 6, where the patient is prompted to click/tap as quickly as possible on a photo 602. The photo 602, a self-relevant stimulus, moves from quadrant to quadrant over time. Following the subject's click/tap, a non-self-relevant stimulus (not shown in FIG. 6) then appears. An example non-self-relevant stimulus is an image of a stranger with a smiling face.

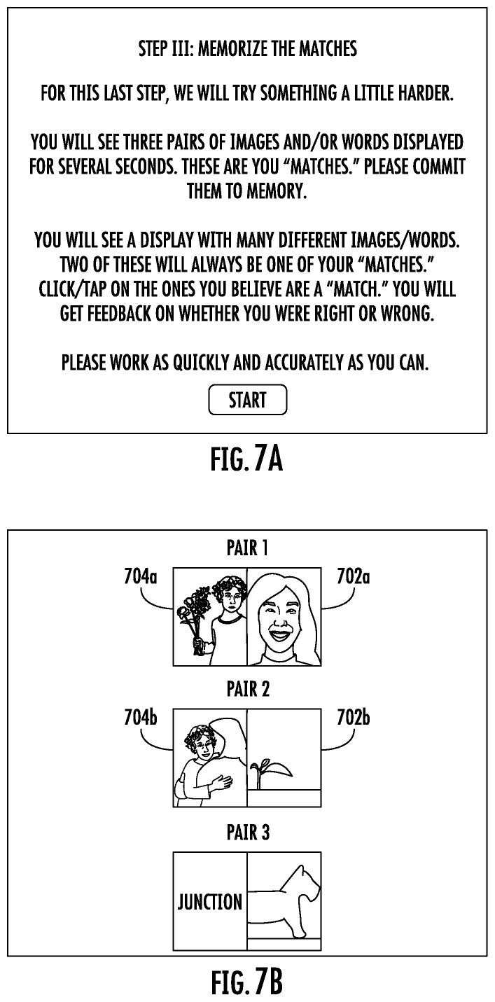

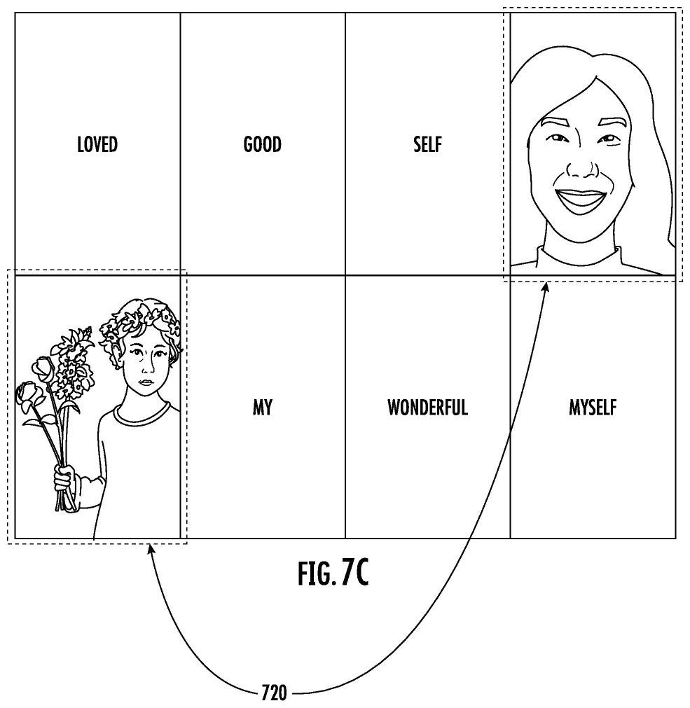

Alternatively or additionally, in some implementations, the respective non-self-relevant stimulus and the respective self-relevant stimulus for each respective conditioning sequence are included in an n×m matrix including a plurality of visual stimuli, where n and m are positive integers. This is shown by FIGS. 7A-7C. In particular, a matrix 710 of visual stimuli is shown in FIG. 7C. In this example, the matrix 710 includes 2 rows and 4 columns of visual stimuli (e.g., words or pictures). In other words, n=2 and m=4 in FIG. 7C. It should be understood that the size of matrix 710 is provided only as an example and may have another size. Prior to presenting the matrix 710, the subject is presented with a plurality of matching pairs of visual stimuli, including at least one pair of non-self-relevant and self-relevant stimuli. This is shown in FIG. 7B. In particular, “Pair 1” in FIG. 7B is a non-self-relevant stimulus 702a (i.e., a photo of a random smiling person, which is a positive trait) and a self-relevant stimulus 704a (i.e., a photo of the subject). “Pair 2” in FIG. 7B is a non-self-relevant stimulus 702b (i.e., a life-affirming or life-relevant photo) and a self-relevant stimulus 704b (i.e., a different photo of the subject). “Pair 3” includes no self-relevant stimulus and is provided to increase the variety and difficulty of the task presentations. During automated neurocognitive training, and as shown in FIG. 7C, the subject is presented with a plurality of visual stimuli matrices including matrix 710 and prompted to respond with a correct selection 720 of the matching non-self-relevant and self-relevant stimuli (e.g., non-self-relevant stimulus 702a and self-relevant stimulus 704a as shown in FIG. 7B).

### Example Methods

The methods of treatment and computer-implemented methods for administering neurocognitive training described herein achieve advantages over conventional technologies by pairing delivery of an antidepressant drug with delivery of automatic neurocognitive training. Specifically, the automated neurocognitive training protocol described herein leverages Pavlovian conditioning in order to promote positive implicit self-representations and self-worth. Such a protocol has been designed to achieve positive results. Additionally, the automated neurocognitive training may include tasks to enhance patient engagement with the training.

An example method for treating a patient having a mental disorder is described herein. This disclosure contemplates that the logical operations of the method described below can be performed using a computing device (e.g., at least one processor and memory such as described with regard to the computing device of FIG. 8). The method includes administering a drug having an antidepressant effect to the patient, and administering a neurocognitive training protocol to the patient during a critical time period following administration of the drug. Additionally, the step of administering the neurocognitive training protocol to the patient includes delivering, using a computing device, a plurality of automated neurocognitive training sessions to the patient. A plurality of conditioning sequences are presented to the patient during delivery of each of the automated neurocognitive training sessions, where each respective conditioning sequence includes a respective non-self-relevant stimulus and a respective self-relevant stimulus.

An example computer-implemented method for administering a neurocognitive training protocol to a patient having a mental disorder is described herein. This disclosure contemplates that the logical operations of the method described below can be performed using a computing device (e.g., at least one processor and memory such as described with regard to the computing device of FIG. 8). The patient has received a drug having an antidepressant effect, and the neurocognitive training protocol is administered during a critical time period following administration of the drug to the patient. The method includes delivering a plurality of automated neurocognitive training sessions to the patient. Additionally, a plurality of conditioning sequences are presented to the patient during delivery of each of the automated neurocognitive training sessions, where each respective conditioning sequence includes a respective non-self-relevant stimulus and a respective self-relevant stimulus.

Another example method for treating a patient having a mental disorder is described herein. This disclosure contemplates that the logical operations of the method described below can be performed using a computing device (e.g., at least one processor and memory such as described with regard to the computing device of FIG. 8). The method includes administering a drug having an antidepressant effect to the patient, and administering a neurocognitive training protocol to the patient during a critical time period following administration of the drug. Additionally, the step of administering the neurocognitive training protocol to the patient includes delivering, using a computing device, a plurality of automated neurocognitive training sessions to the patient. Each respective automated neurocognitive training session includes presenting an n×m matrix including a plurality of visual stimuli to the patient, and receiving the patient's selection of the first matching pair of non-self-relevant and self-relevant visual stimuli. The plurality of visual stimuli include a first matching pair of non-self-relevant and self-relevant visual stimuli. Additionally, in some implementations, each respective automated neurocognitive training session further includes presenting a second matching pair of non-self-relevant and self-relevant visual stimuli to the patient, and presenting a task to the patient following presentation of the second matching pair of non-self-relevant and self-relevant visual stimuli. Optionally, each respective automated neurocognitive training session further includes presenting a self-relevant visual stimulus to the patient, presenting a task to the patient following presentation of the self-relevant stimulus, and presenting a positive self-relevant visual stimulus following successful completion of the task by the patient.

Another example method for treating a patient having a mental disorder is described herein. This disclosure contemplates that the logical operations of the method described below can be performed using a computing device (e.g., at least one processor and memory such as described with regard to the computing device of FIG. 8). The method includes: administering a drug having an antidepressant effect to the patient; and administering a neurocognitive training protocol to the patient during a critical time period following administration of the drug. The step of administering the neurocognitive training protocol to the patient includes delivering, using a computing device, a plurality of automated neurocognitive training sessions to the patient, wherein a plurality of conditioning sequences are presented to the patient during delivery of each of the automated neurocognitive training sessions.

An example method for conditioning a therapeutic association in a patient is described herein. This disclosure contemplates that the logical operations of the method described below can be performed using a computing device (e.g., at least one processor and memory such as described with regard to the computing device of FIG. 8). The method includes: administering a neuroplasticity-enhancing treatment to the patient; and administering a neurocognitive training protocol to the patient during a critical time period following administration of the neuroplasticity-enhancing treatment. The step of administering the neurocognitive training protocol to the patient includes delivering, using a computing device, one or more conditioning sequences configured to condition a therapeutic association to the patient. Non-limiting examples of combinations of neuroplasticity-enhancing treatments and neurocognitive training protocols are described below. This disclosure contemplates that any one of the example neuroplasticity-enhancing treatments may be combined with any one of the example neurocognitive training protocol in combination in order to condition a therapeutic association.

In some implementations, the neuroplasticity-enhancing treatment is a neuromodulatory intervention. Neuromodulatory interventions include non-pharmacological interventions such as transcranial magnetic stimulation, transcranial direct current stimulation, transcranial focused ultrasound, or other neuromodulatory manipulation. Alternatively or additionally, in other implementations, the neuroplasticity-enhancing treatment is a drug. As described herein, the drug may be an antidepressant, and optionally a rapid-acting antidepressant such as ketamine. It should be understood that antidepressants are only provided as an example and that other drugs or neuroplasticity-enhancing agents may be delivered.

Additionally, in some implementations, the neurocognitive training protocol includes delivery of automated neurocognitive training sessions as described herein, e.g., a plurality of conditioning sequences that pair self-relevant and non-self-relevant stimuli. In other implementations, the neurocognitive training protocol includes delivery of automated neurocognitive training sessions that may include, but are not limited to, therapeutic pairings targeting a variety of mental health concerns. Such therapeutic pairings may include a stimulus where approaching the stimulus or activity is desired [e.g., feared (phobic) stimulus; healthy and/or valued activities or scenarios that are presently avoided; cues related to a future-oriented mindset] and a respective positive stimulus. This neurocognitive training can be used to condition approach towards a feared (phobic) stimulus, healthy and/or valued activity or scenario that is presently avoided, or cue related to a future-oriented mindset. Alternatively, such therapeutic pairings may include an undesirable stimulus where avoiding the stimulus or activity is desired (e.g., addiction-related craving cue; self-injury-related cue) and a respective negative stimulus. This neurocognitive training can be used to condition increased resistance to or avoidance of a craving (addiction-related), self-injury-related, or other undesirable cue.

### Example Computing Device

It should be appreciated that the logical operations described herein with respect to the various figures may be implemented (1) as a sequence of computer implemented acts or program modules (i.e., software) running on a computing device (e.g., the computing device described in FIG. 8), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 8, an example computing device 800 upon which the methods described herein may be implemented is illustrated. It should be understood that the example computing device 800 is only one example of a suitable computing environment upon which the methods described herein may be implemented. Optionally, the computing device 800 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 800 typically includes at least one processing unit 806 and system memory 804. Depending on the exact configuration and type of computing device, system memory 804 may be volatile (such as random access memory (RAM)), non-volatile (such as read-only memory (ROM), flash memory, etc.), or some combination of the two. This most basic configuration is illustrated in FIG. 8 by dashed line 802. The processing unit 806 may be a standard programmable processor that performs arithmetic and logic operations necessary for operation of the computing device 800. The computing device 800 may also include a bus or other communication mechanism for communicating information among various components of the computing device 800.

Computing device 800 may have additional features/functionality. For example, computing device 800 may include additional storage such as removable storage 808 and non-removable storage 810 including, but not limited to, magnetic or optical disks or tapes. Computing device 800 may also contain network connection(s) 816 that allow the device to communicate with other devices. Computing device 800 may also have input device(s) 814 such as a keyboard, mouse, touch screen, etc. Output device(s) 812 such as a display, speakers, printer, etc. may also be included. The additional devices may be connected to the bus in order to facilitate communication of data among the components of the computing device 800. All these devices are well known in the art and need not be discussed at length here.

The processing unit 806 may be configured to execute program code encoded in tangible, computer-readable media. Tangible, computer-readable media refers to any media that is capable of providing data that causes the computing device 800 (i.e., a machine) to operate in a particular fashion. Various computer-readable media may be utilized to provide instructions to the processing unit 806 for execution. Example tangible, computer-readable media may include, but is not limited to, volatile media, non-volatile media, removable media and non-removable media implemented in any method or technology for storage of information such as computer readable instructions, data structures, program modules or other data. System memory 804, removable storage 808, and non-removable storage 810 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 806 may execute program code stored in the system memory 804. For example, the bus may carry data to the system memory 804, from which the processing unit 806 receives and executes instructions. The data received by the system memory 804 may optionally be stored on the removable storage 808 or the non-removable storage 810 before or after execution by the processing unit 806.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

A randomized controlled trial to test whether rapid mood relief during a “window of opportunity” after ketamine infusion can be extended and/or enhanced, by reinforcing (“conditioning in”) helpful, positive views of oneself through fully automated neurocognitive training (NT) software was completed. Automated N T is also referred to herein as automated self-association training (ASAT). A critical gap in previous efforts to develop NT treatments, which this study filled, was to initiate NT synergistically during a key clinical window of opportunity (immediately following ketamine infusion), when improved mood and enhanced neuroplasticity are expected, to consolidate beneficial processing patterns and increase and prolong ketamine's rapid mood effects.

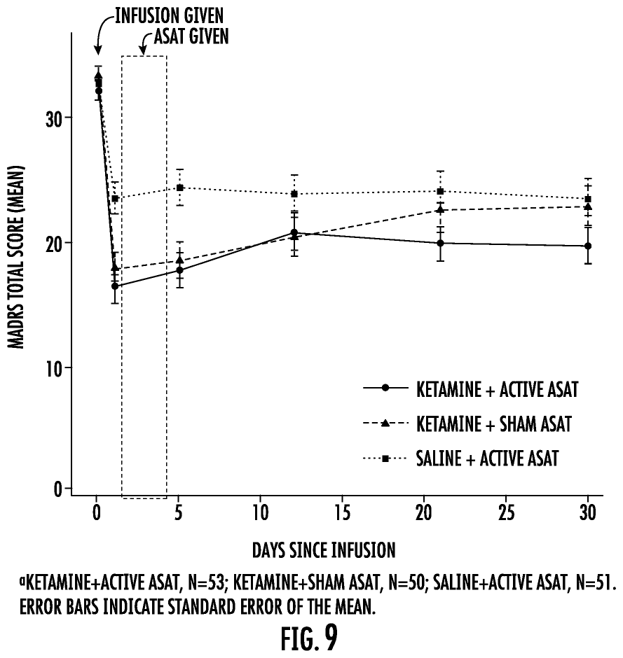

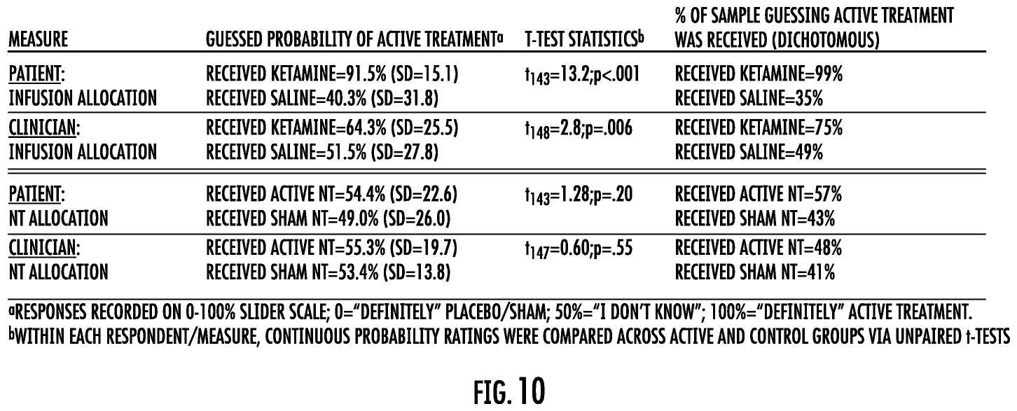

The results from this double-blind, randomized controlled trial confirmed hypotheses. Ketamine rapidly reduced depression scores at 24 hours post-infusion (relative to a saline control infusion) among patients with treatment-resistant depression (i.e., patients who had previously failed to respond to first-line antidepressant medications). A brief (2.5 hours total, eight 20 min sessions spread over 4 days), fully automated, computer-based NT intervention, designed to condition in positive self-representations, or a sham/placebo version of NT was then initiated. Over a 30-day acute post-infusion phase, the combination of ketamine+active NT (ketamine+active ASAT in FIG. 9) produced a stable, enduring decrease in depression relative to saline+NT (saline+active ASAT in FIG. 9). By contrast, ketamine+Sham N T (ketamine+sham ASAT in FIG. 9) produced an initial decrease in depression that waned linearly over the 30-day window, becoming non-significant by Day 12 post-infusion. In an ongoing naturalistic followup, the ketamine+NT arm showed continued benefits on self-reported depression 3-months post-infusion (see Example 9).

In another ongoing randomized controlled trial, this synergistic combination (ketamine+computer-based NT) is tested among hospitalized inpatients immediately following a suicide attempt, in an effort to prevent future suicidal thoughts, suicide reattempts, and completed suicides among this very high-risk group.

In summary, brief, synergistic treatment, combining ketamine infusion with a fully automated cognitive training software, urgently brought relief and then efficiently extended this relief among treatment-resistant depressed patients, by exploiting a window of opportunity via safe, low-cost, portable techniques. There are wide potential applications for this treatment strategy, given that intravenous ketamine is very routinely available and used widely in hospital settings as well as in specialty community-based clinics throughout the world; and the fully automated cognitive training software is extremely efficient, simple, easy, and feasible to administer and to complete, in both clinical settings and in the patient's home.

### Example 2

### INTRODUCTION

Depression is one of the most prevalent and costly mental health conditions [Kessler R C, Chiu W T, Demler O, Merikangas K R, Walters E E: Prevalence, severity, and comorbidity of 12-month DSM-IV disorders in the National Comorbidity Survey Replication. Arch Gen Psychiatry. 2005; 62(6):617-27], with a public disease burden of staggering proportions [Kessler R C: The global burden of anxiety and mood disorders: putting the European Study of the Epidemiology of Mental Disorders (ESEMeD) findings into perspective. J Clin Psychiatry. 2007; 68 Suppl 2:10-9]. While efficacious treatments have been available for decades, remission rates are low, relapse rates are high, and disorder prevalence rates remain notably consistent or increased, with only 12.7% of patients receiving minimally adequate treatment [Wang P S, Lane M, Olfson M, Pincus H A, Wells K B, Kessler R C: Twelve-month use of mental health services in the United States: results from the National Comorbidity Survey Replication. Arch Gen Psychiatry. 2005; 62(6):629-40]. In recent years, two potential breakthroughs have ignited hopes for turning a corner in the clinical management of this disabling disorder. First, rapid-acting pharmacological agents-notably, intravenous ketamine [Xu Y, Hackett M, Carter G, Loo C, Galvez V, Glozier N, Glue P, Lapidus K, McGirr A, Somogyi A A, Mitchell P B, Rodgers A: Effects of low-dose and very low-dose dose ketamine among patients with major depression: a systematic review and meta-analysis. Int J Neuropsychopharmacol. 2015; Newport D J, Carpenter L L, McDonald W M, Potash J B, Tohen M, Nemeroff C B, Treatments APACoRTFoNBa: Ketamine and Other NMDA Antagonists: Early Clinical Trials and Possible Mechanisms in Depression. Am J Psychiatry. 2015; 172(10):950-66; Caddy C, Amit B H, McCloud T L, Rendell J M, Furukawa T A, McShane R, Hawton K, Cipriani A: Ketamine and other glutamate receptor modulators for depression in adults. Cochrane Database Syst Rev. 2015; September 23(9):CD011612]—have shown promise in dramatically reducing symptoms within the space of 2-24 hours, overcoming the sluggish response to conventional therapies (e.g., 4-6 week delays) which prolongs suffering, creates non-compliance issues, and fails to address urgent clinical needs (e.g., suicidality and other psychiatric crises). Second, intervention development has increasingly sought to take advantage of technology to increase patient access, reduce cost, and minimize aversive consequences through the use of automated, computer-based procedures [Mohr D C, Burns M N, Schueller S M, Clarke G, Klinkman M: Behavioral intervention technologies: evidence review and recommendations for future research in mental health. Gen Hosp Psychiatry. 2013; 35(4):332-8]. The past decade has seen growing interest in automated, mechanistic NT treatments, designed to modulate affective processing patterns directly in order to reduce symptoms [Siegle G J, Price R B, Jones N, Ghinassi F, Painter T, Thase M E: You gotta work at it: Pupillary indices of task focus are prognostic for response to a neurocognitive intervention for depression. Clin Psychol Sci. 2014; 2(4):455-71]; but clinical effects, though showing potential, are unreliable, particularly in acutely depressed patients [Mogoase C, David D, Koster E H W: Clinical Efficacy of Attentional Bias Modification Procedures: An Updated Meta-analysis. J Clin Psychol. 2014; 70:1133-57; Motter J N, Pimontel M A, Rindskopf D, Devanand D P, Doraiswamy P M, Sneed J R: Computerized cognitive training and functional recovery in major depressive disorder: A meta-analysis. J Affect Disord. 2016; 189:184-91]. The study described below is aimed to synergistically combine the benefits of these two approaches.

The rapidity of ketamine's effects offers the promise of a breakthrough in the way that depression is managed, but this promise remains unfulfilled to date. Quickly dissipating effects [Walter M, Li S, Demenescu L R: Multistage drug effects of ketamine in the treatment of major depression. Eur Arch Psychiatry Clin Neurosci. 2014; 264 Suppl 1:S55-65], coupled with substantial concerns regarding safety and feasibility of repeated infusions [Schatzberg A F: A word to the wise about ketamine. Am J Psychiatry. 2014; 171(3):262-4; Price R B: From Mice to Men: Can Ketamine Enhance Resilience to Stress?Biol Psychiatry. 2016; 79(9):e57-9], limit ketamine's clinical impact. While substantial advancements have recently been made in understanding ketamine's molecular mechanisms of action [Zanos P, Moaddel R, Morris P J, Georgiou P, Fischell J, Elmer G I, Alkondon M, Yuan P, Pribut H J, Singh N S, Dossou K S, Fang Y, Huang X P, Mayo C L, Wainer I W, Albuquerque E X, Thompson S M, Thomas C J, Zarate C A, Jr., Gould T D: NMDAR inhibition-independent antidepressant actions of ketamine metabolites. Nature. 2016; 533(7604):481-6; Malinow R: Depression: Ketamine steps out of the darkness. Nature. 2016; 533(7604):477-8] and developing strategies to extend these effects pharmacologically, translation to safe, effective, novel pharmacology can be slow and uncertain. Furthermore, long-term pharmacological intervention is unlikely to be beneficial, feasible, and appealing to every patient [Bockting C L, ten Doesschate M C, Spijker J, Spinhoven P, Koeter M W, Schene A H, group Ds: Continuation and maintenance use of antidepressants in recurrent depression. Psychother Psychosom. 2008; 77(1):17-26]. A large majority of patients (e.g., up to 91% of depressed patients enrolling in drug trials [Steidtmann D, Manber R, Arnow B A, Klein D N, Markowitz J C, Rothbaum B O, Thase M E, Kocsis J H: Patient treatment preference as a predictor of response and attrition in treatment for chronic depression. Depress Anxiety. 2012; 29(10):896-905]) consistently express preference for behavioral or combined behavioral/pharmacological treatments [Huijbers M J, Spinhoven P, van Schaik D J, Nolen W A, Speckens A E: Patients with a preference for medication do equally well in mindfulness-based cognitive therapy for recurrent depression as those preferring mindfulness. J Affect Disord. 2016; 195:32-; van Schaik D J, Klijn A F, van Hout H P, van Marwijk H W, Beekman A T, de Haan M, van Dyck R: Patients' preferences in the treatment of depressive disorder in primary care. Gen Hosp Psychiatry. 2004; 26(3):184-9], in part reflecting the belief that such treatments will introduce new learning that will solve the core problem and instantiate lasting change [van Schaik D J, Klijn A F, van Hout H P, van Marwijk H W, Beekman A T, de Haan M, van Dyck R: Patients' preferences in the treatment of depressive disorder in primary care. Gen Hosp Psychiatry. 2004; 26(3):184-9]. Antidepressant pharmacological regimens are challenging to maintain in the long term due to patient discontinuation [Bockting C L, ten Doesschate M C, Spijker J, Spinhoven P, Koeter M W, Schene A H, group Ds: Continuation and maintenance use of antidepressants in recurrent depression. Psychother Psychosom. 2008; 77(1):17-26] and the rarity of follow-up opportunities in community practice [Simon G E, Von Korff M, Rutter C M, Peterson Da: Treatment process and outcomes for managed care patients receiving new antidepressant prescriptions from psychiatrists and primary care physicians. Arch Gen Psychiatry. 2001; 58:395-401].

Ketamine is posited to reverse the molecular signature of depression via synaptogenic and neuroplasticity effects at the molecular level [Zanos P, Moaddel R, Morris P J, Georgiou P, Fischell J, Elmer G I, Alkondon M, Yuan P, Pribut H J, Singh N S, Dossou K S, Fang Y, Huang X P, Mayo C L, Wainer I W, Albuquerque E X, Thompson S M, Thomas C J, Zarate C A, Jr., Gould T D: NMDAR inhibition-independent antidepressant actions of ketamine metabolites. Nature. 2016; 533(7604):481-6; Abdallah C G, Sanacora G, Duman R S, Krystal J H: Ketamine and rapid-acting antidepressants: a window into a new neurobiology for mood disorder therapeutics. Annu Rev Med. 2015; 66:509-23; Duman R S, Aghajanian G K, Sanacora G, Krystal J H: Synaptic plasticity and depression: new insights from stress and rapid-acting antidepressants. Nat Med. 2016; 22(3):238-49]. Based on prior work, it was hypothesized [Price R B, Duman R: Neuroplasticity in cognitive and psychological mechanisms of depression: An integrative model. Mol Psychiatry. 2020; 25(3):530-43; Wilkinson S T, Holtzheimer P E, Gao S, Kirwin D S, Price R B: Leveraging Neuroplasticity to Enhance Adaptive Learning: The Potential for Synergistic Somatic-Behavioral Treatment Combinations to Improve Clinical Outcomes in Depression. 2019; 85(6):454-65] that these molecular effects may produce a corresponding neurocognitive shift, in which can extend rapid mood relief during a window of opportunity using behavioral learning-based approaches—for example, by reinforcing adaptive patterns of cognition through neurocognitive training (NT). This study was designed to initiate NT synergistically during a key clinical window of opportunity—i.e. 1 day following a ketamine infusion, when euthymic mood and enhanced plasticity were expected—in order to consolidate beneficial processing patterns and prolong ketamine's rapid mood effects.

Consistent with rising momentum around the creation of synergistic bio-behavioral approaches to psychiatric management (including both ketamine [Dakwar E, Levin F R, Hart C L, Basaraba C N, Choi C J, Pavlicova M, Nunes E V: A single ketamine infusion combined with motivational enhancement therapy for alcohol use disorder: A randomized, midazolam-controlled pilot trial. Am J Psychiatry. 2020; 177:125-33; Dakwar E, Nunes E V, Hart C L, Foltin R W, Mathew S J, Carpenter K M, Choi C J J, Basaraba C N, Pavlicova M, Levin F R: A Single Ketamine Infusion Combined With Mindfulness-Based Behavioral Modification to Treat Cocaine Dependence: A Randomized Clinical Trial. Am J Psychiatry. 2019:appiajp201918101123; Wilkinson S T, Rhee T G, Joormann J, Webler R, Ortiz Lopez M, Kitay B, Fasula M, Elder C, Fenton L, Sanacora G: Cognitive Behavioral Therapy to Sustain the Antidepressant Effects of Ketamine in Treatment-Resistant Depression: A Randomized Clinical Trial. Psychother Psychosom. 2021; 90(5):318-27] and other “psychoplastogenic” drugs [Vargas M V, Meyer R, Avanes A A, Rus M, Olson D E: Psychedelics and Other Psychoplastogens for Treating Mental Illness. Front Psychiatry. 2021; 12:727117]), the study was designed to push the boundaries of how rapid-acting antidepressant drug agents could be used clinically, i.e. as short-term cognitive flexibility enhancers to promote enduring learning that would be antithetical to the depressed state. Ketamine infusion was used as the first step in a treatment protocol designed to foster relief that would be both rapid and durable, simultaneously taking advantage of technology's potential for low-cost, portable, safe, dissemination-ready intervention. Consistent with findings suggesting behavioral treatments can prevent depression relapse even in the absence of ongoing care [Bockting C L, ten Doesschate M C, Spijker J, Spinhoven P, Koeter M W, Schene A H, group Ds: Continuation and maintenance use of antidepressants in recurrent depression. Psychother Psychosom. 2008; 77(1):17-26; Bockting C L, Schene A H, Spinhoven P, Koeter M W, Wouters L F, Huyser J, Kamphuis J H: Preventing relapse/recurrence in recurrent depression with cognitive therapy: a randomized controlled trial. J Consult Clin Psychol. 2005; 73(4):647-57; Hollon S D, Stewart M O, Strunk D: Enduring effects for cognitive behavior therapy in the treatment of depression and anxiety. 2006; 57:285-315; Hollon S D, DeRubeis R J, Shelton R C, Amsterdam J D, Salomon R M, O'Reardon J P, Lovett M L, Young P R, Haman K L, Freeman B B, Gallop R: Prevention of relapse following cognitive therapy vs medications in moderate to severe depression. Arch Gen Psychiatry. 2006; 62:417-22], and preliminary evidence that brief NT approaches can likewise have enduring and clinically meaningful effects [Siegle G J, Price R B, Jones N, Ghinassi F, Painter T, Thase M E: You gotta work at it: Pupillary indices of task focus are prognostic for response to a neurocognitive intervention for depression. Clin Psychol Sci. 2014; 2(4):455-71; Franklin J C, Fox K R, Franklin C R, Kleiman E M, Ribeiro J D, Jaroszewski A C, Hooley J M, Nock M K: A brief mobile app reduces nonsuicidal and suicidal self-injury: Evidence from three randomized controlled trials. J Consult Clin Psychol. 2016; 84(6):544-57], it was hypothesized that the introduction and facilitation of new learning during a ketamine-induced window-of-opportunity would provide an efficient path to relief that could be both rapid and enduring. In this first-of-its-kind study, the active/active combination of ketamine+automated Neurocognitive Training (NT) as described herein was compared, in a randomized, double-blind, parallel arm design, to control conditions that included each intervention component in the absence of the other (i.e., saline+active NT; ketamine+sham NT). This enabled a direct test of hypothesized synergy between ketamine and NT, in an effort to generate antidepressant action that was both rapid and enduring.

### Example 3

### Methods

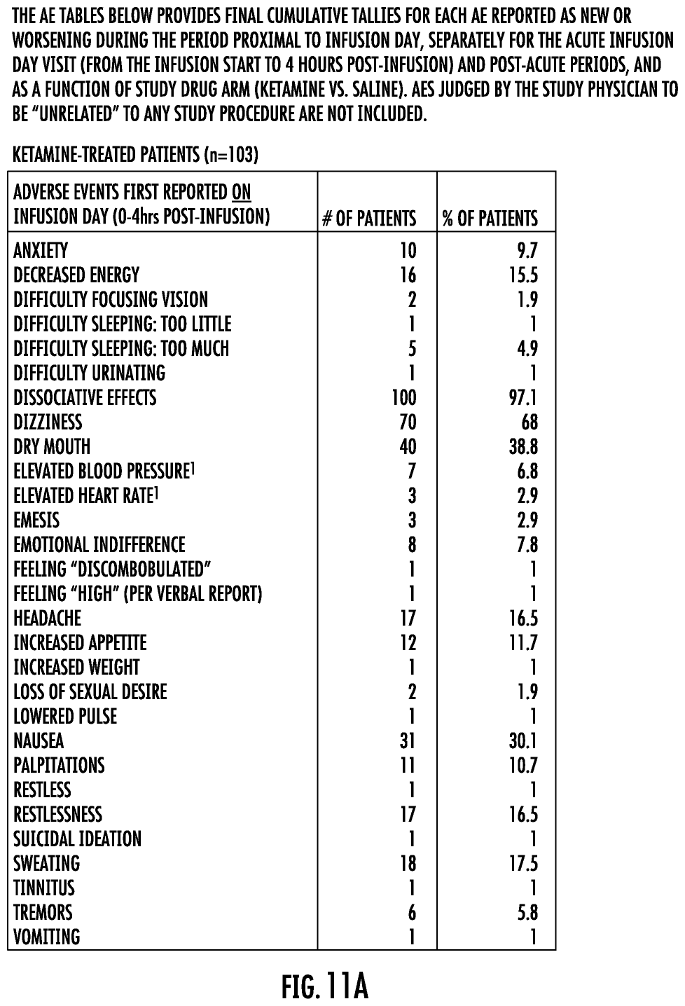

Participants. To achieve the initial recruitment target of n=150 patients, the study enrolled and randomized n=154 patients, continuing enrollment until a final sample was acquired that included the full target of n=150 patients who completed, at a minimum, (1) an infusion day, (2) a 24-hour assessment visit, and (3) ≥75% of intended Cognitive Training (active or sham NT) visits. All n=154 randomized patients were included in the reported intent-to-treat analyses. In particular, 154 adult (age 18-60) patients reporting moderate-to-severe levels of depression [Montgomery-Asberg Depression Rating Scale (MADRS; [Montgomery S A, Asberg M: A new depression scale designed to be sensitive to change. Br J Psychiatry. 1979; 134:382-9]) score 25], lower-than-normative self-reported self-esteem [i.e., scoring outside of 1SD from the normative mean on the Cognitive Triad Inventory [Beckham E E, Leber W R, Watkins J T, Boyer J L, Cook J B: Development of an instrument to measure Beck's cognitive triad: the Cognitive Triad Inventory. J Consult Clin Psychol. 1986; 54(4):566-7]“self” subscale [Anderson K W, Skidmore J R: Empirical analysis of factors in depressive cognition: the cognitive triad inventory. J Clin Psychol. 1995; 51(5):603-9; Possel P: Cognitive Triad Inventory (CTI): psychometric properties and factor structure of the German translation. J Behav Ther Exp Psychiatry. 2009; 40(2):240-7] and/or the Rosenberg Self-Esteem Scale], and at least one failed, adequate trial of an FDA-approved antidepressant medication in the current depressive episode (per Antidepressant Treatment Response Questionnaire; ATRQ [Chandler G M, losifescu DV, Pollack M H, Targum S D, Fava M: RESEARCH: Validation of the Massachusetts General Hospital Antidepressant Treatment History Questionnaire (ATRQ). CNS Neurosci Ther. 2010; 16(5):322-5]), were recruited and randomized. Any existing depression treatment regimens were required to be stably maintained beginning 4 weeks prior to screening and throughout the trial. See FIG. 10, Table S1 and Examples 6-8 below for sample characteristics.

To achieve the initial recruitment target of a sample of 150 patients, the study enrolled and randomized 154 patients, continuing enrollment until a final sample was acquired that included the full target of 150 patients who completed, at a minimum, an infusion day, a 24-hour assessment visit, and ≥75% of intended training (active or sham ASAT) visits, and were thus considered to have received their ASAT allocation. All but two participants who surpassed this 75% threshold for their ASAT allocation received the full, eight session dose of intended ASAT visits, per protocol (N=148 for full, per-protocol completers who received all allocated ASAT sessions). Thus, 96% of randomized participants were fully adherent with all intervention sessions, and 97% of all intended ASAT sessions were delivered.

A three-arm study design was selected to allocate all available resources toward the active/active (ketamine+ASAT) treatment arm relative to two crucial comparator groups, comprising each intervention component in the absence of the other—a conservative test of the active/active biobehavioral intervention's combined impact. An inactive/inactive (no-treatment) arm was forgone in order to maximize statistical power for these more conservative and critical comparisons. An a priori power analysis was based on a principal interest in clinically meaningful, moderate (or larger) effects. The target sample sizes were selected to yield 80% power to detect moderate effects based on infusion phase comparisons of 100 ketamine and 50 saline patients (d≥0.49 and r≥0.31 using an alpha of 0.05), and based on comparisons of 50 patients in each of the ketamine+ASAT, ketamine+sham, and saline+ASAT conditions during the ASAT phase, where effects of d≥20.57 would be detectable with 80% power (using an alpha of 0.05). Power for primary mixed-effect analyses comparing treatment groups, which increases with additional repeated measures, was anticipated to be higher than for these simplified, single-point contrasts, making these power calculations conservative.

Assessment Timeline. An experienced, Master's level clinical rater (CRS) administered the MADRS, the study's pre-specified primary endpoint throughout the acute (30-day) study period, to assess overall depression severity. The measure was repeated at screening (used for eligibility determinations only), pre-infusion baseline (infusion −1 hour; the clinical rater then left and was not present during any intervention procedure), infusion +24 hours (prior to the first session of active/sham NT), +5, +12, +21, and +30 days. A 1-year exploratory naturalistic follow-up, utilizing remote assessments acquired strictly through a distinct self-report symptom scale (QIDS-SR [Rush A J, Trivedi M H, Ibrahim H M, Carmody T J, Arnow B, Klein D N, Markowitz J C, Ninan P T, Kornstein S, Manber R, Thase M E, Kocsis J H, Keller M B: The 16-Item Quick Inventory of Depressive Symptomatology (QIDS), Clinician Rating (QIDS-C) and Self-Report (QIDS-SR): A Psychometric evaluation in patients with chronic major depression. Biol Psychiatry. 2003; 54:573-83]), remains ongoing and thus these data are not included in the present report. This study also contemplates including and analyzing neurocognitive (e.g., neuroimaging; performance-based) assessments of ketamine's impact. Adequacy of both patient and rater blinding, for both infusion and NT allocation, was assessed by self-report using a 0-100 sliding scale (0=definitely placebo/sham; 50=1 don't know; 100=definitely ketamine/active) at infusion+30 days.

Interventions. A randomization table using permuted blocks of 3 or 6, stratified by (1) biological sex at birth and (2) modest vs. severe treatment resistance (severe=≥3 FDA-approved antidepressant trials in episode, per ATRQ), was generated by an independent statistical consultant at study start and maintained by the study pharmacist, who, at the point of randomization, conveyed the NT allocation (but not the infusion allocation) to a single study staff member who was assigned to that patient. This single staff member was tasked with opening the correct computer program to deliver the NT sessions to that patient, but was not involved in, nor present for, any outcome ratings. Blinding for all other study personnel was maintained until after the final patient's Day 30 assessment was complete.

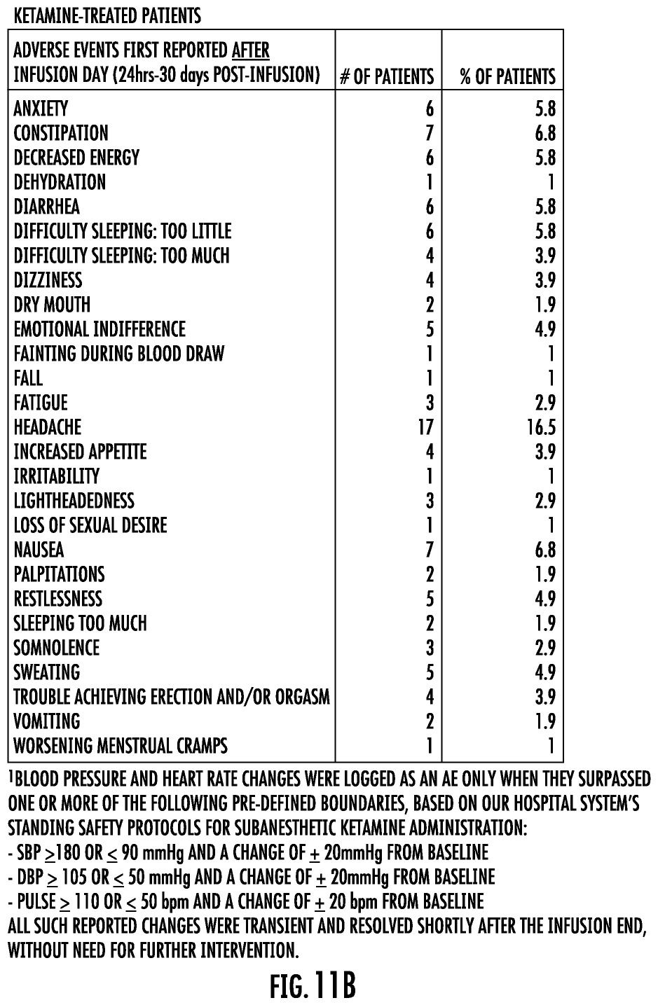

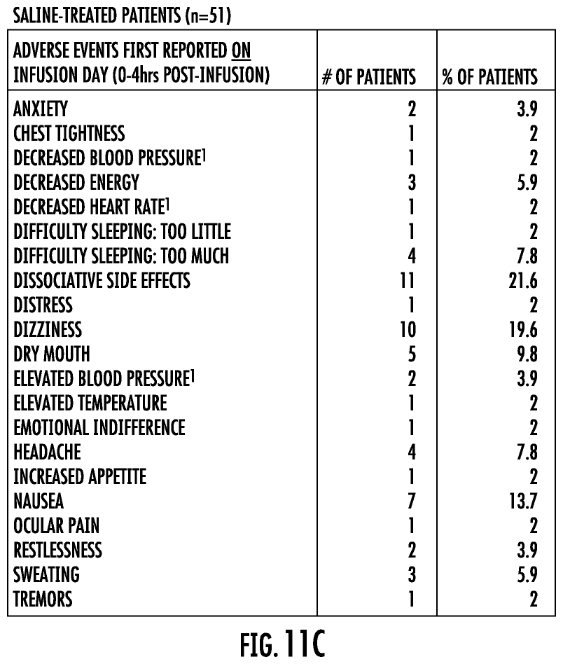

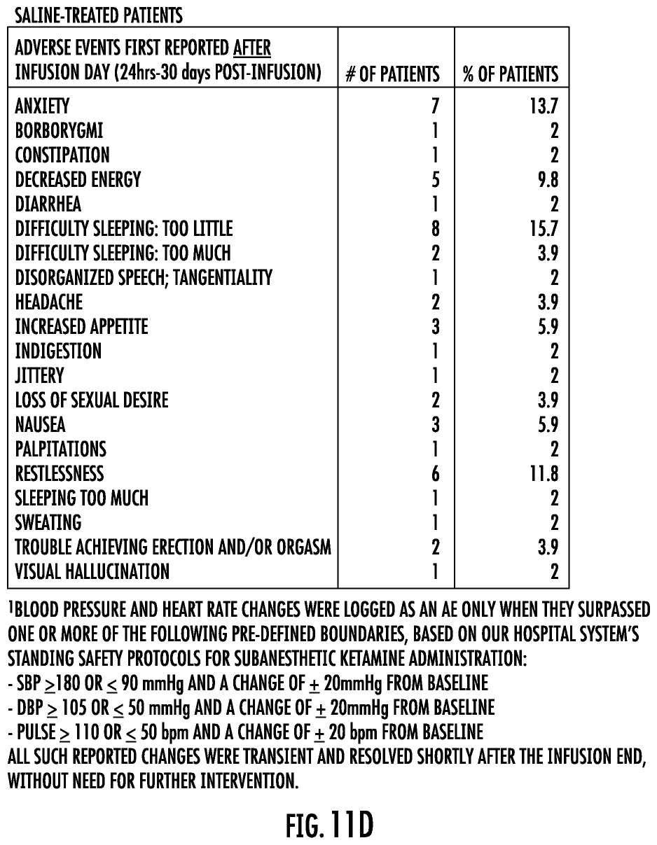

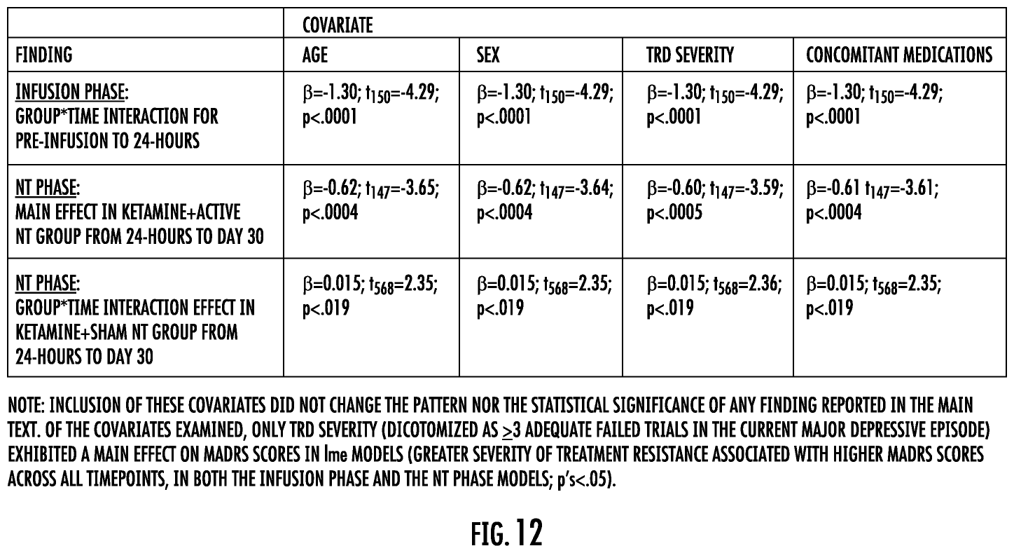

Infusion. Participants were randomized 2:1 to ketamine (0.5 mg/kg over 40 min) or saline (50 ml 0.9% Sodium Chloride over 40 min) infusion. All infusions were administered by blinded, licensed nurses in a medical hospital setting with a linked ACLS-certified team, with blinded study physician (RHH) oversight and safety/adverse event monitoring sustained for 4-hours post-infusion. Adverse events (FIG. 11A-11D, Table S2) were mild-to-moderate, time-limited, and consistent with the safety profile described in numerous previous trials using identical infusion procedures.

Cognitive Training. Drawing on previous work utilizing ‘evaluative conditioning’ to influence participants' preferences and ‘liking,’ [De Houwer J, Thomas S, Baeyens F: Associative learning of likes and dislikes: a review of 25 years of research on human evaluative conditioning. Psychol Bull. 2001; 127(6):853-69; Martijn M V, Roefs A, Huijding J, Jansen A: Increasing body satisfaction of body concerned women through evaluative conditioning using social stimuli. Health Psychol. 2010; 29(5):514-20] active NT (also referred to herein as active ASAT) was designed to leverage Pavlovian conditioning in order to promote positive implicit self-representations and self-worth. Across each of eight 15-20 minute sessions (delivered twice daily for 4 consecutive days, spaced by a ≥20 min inter-session interval, initiated 1 day post-infusion, after the 24-hour post-infusion assessment), both verbal and pictorial stimulus pairings were presented, both supraliminally (250 ms) and subliminally (12 ms), to practice and reinforce implicit associations between positive traits [e.g., ‘sweet’, ‘attractive’, photos of smiling actors; the unconditioned stimuli (US)] and self-referential stimuli (e.g., ‘I’, participant headshots; the conditioned stimuli (CS)]. Incidental tasks such as a ‘lexical decision’ task, indicating whether each target is a real word or a random letter string, and a rapid mouse-tracking task (clicking as fast as possible on the position of stimuli) were used to enhance engagement with the task and to promote both semantic and visual processing of stimuli. Similar forms of ‘evaluative conditioning’ have been found to alter some measures of implicit self-esteem in healthy participants [Martijn M V, Roefs A, Huijding J, Jansen A: Increasing body satisfaction of body concerned women through evaluative conditioning using social stimuli. Health Psychol. 2010; 29(5):514-20; Dijksterhuis A: I like myself but I don't know why: enhancing implicit self-esteem by subliminal evaluative conditioning. J Pers Soc Psychol. 2004; 86(2):345-55; Baccus J R, Baldwin M W, Packer D J: Increasing implicit self-esteem through classical conditioning. Psychol Sci. 2004; 15(7):498-502], mood reactivity to stress [Dijksterhuis A: I like myself but I don't know why: enhancing implicit self-esteem by subliminal evaluative conditioning. J Pers Soc Psychol. 2004; 86(2):345-55], and pathological behaviors (anxious avoidance [Clerkin E M, Teachman B A: Training implicit social anxiety associations: an experimental intervention. J Anxiety Disord. 2010; 24(3):300-8], self-injury [Franklin J C, Fox K R, Franklin C R, Kleiman E M, Ribeiro J D, Jaroszewski A C, Hooley J M, Nock M K: A brief mobile app reduces nonsuicidal and suicidal self-injury: Evidence from three randomized controlled trials. J Consult Clin Psychol. 2016; 84(6):544-57]). Sham N T consisted of the exact same computer tasks, but with neutral rather than positive US and non-self-relevant CS (words related to ‘others’, pictures of gender-matched strangers), designed to eliminate the possibility of unintended self-referential or negative/iatrogenic learning, while providing a credible “brain-training” paradigm that controlled for all non-specific factors and facilitated patient blinding.

Statistical Analysis. Intent-to-treat, linear mixed effects regression models were applied with continuous MADRS scores as the outcome; days since infusion as a random, continuous within-subject effect; and treatment allocation as a fixed, between-subjects effect. Separate models were designed to test two discrete hypothesized linear patterns, based on: (1) the Infusion Phase of intervention (ketamine vs. saline), using pre-infusion and 24-hour assessments only (collected prior to onset of active/sham NT); and (2) the NT Phase of intervention (3-way split: saline+NT vs. ketamine+sham vs. ketamine+NT), using 24-hour through Day 30 assessment points. In the infusion phase, it was hypothesized rapid symptom decreases in the ketamine arm (relative to saline). In the NT Phase, it was hypothesized a linear increase in symptoms from 24-hours to Day 30 in the ketamine+sham arm (group*time interaction relative to saline+NT), consistent with the 1-2 week duration of improvements provided by a single infusion of ketamine in isolation in prior published work [Xu Y, Hackett M, Carter G, Loo C, Galvez V, Glozier N, Glue P, Lapidus K, McGirr A, Somogyi A A, Mitchell P B, Rodgers A: Effects of low-dose and very low-dose dose ketamine among patients with major depression: a systematic review and meta-analysis. Int J Neuropsychopharmacol. 2015; Newport D J, Carpenter L L, McDonald W M, Potash J B, Tohen M, Nemeroff C B, Treatments APACoRTFoNBa: Ketamine and Other NMDA Antagonists: Early Clinical Trials and Possible Mechanisms in Depression. Am J Psychiatry. 2015; 172(10):950-66; Caddy C, Amit B H, McCloud T L, Rendell J M, Furukawa T A, McShane R, Hawton K, Cipriani A: Ketamine and other glutamate receptor modulators for depression in adults. Cochrane Database Syst Rev. 2015; September 23(9):CD011612]; and a stable (main) effect extending from 24-hours to Day 30 in the ketamine+active NT arm (relative to saline+NT). NT Phase models utilized the saline+NT arm as the reference group for hypothesis tests, and included pre-infusion MADRS scores as a covariate. All models included a random intercept and slope for participant to model patient-level trajectories over time and automatically account for missing data (5.1% of all intended observations). For interpretability, continuous variables were standardized and dichotomous variables were coded as 0.5 and −0.5. Standardized coefficients (p*) and odds ratios (OR) with 95% profile likelihood confidence intervals are reported. Analyses were performed using R version 3.6.

For descriptive and clinical characterization purposes only, dichotomous outcomes were defined for responders (≥50% decrease from pre-infusion MADRS baseline) and remitters (MADRS≤9 [Hawley C J, Gale T M, Sivakumaran T, Hertfordshire Neuroscience Research g: Defining remission by cut off score on the MADRS: selecting the optimal value. J Affect Disord. 2002; 72(2):177-84]) at each post-infusion assessment point.

Sensitivity analyses (see Examples 6-8) probed for the robustness of findings when including the following covariates selected a priori: sex, age, treatment-resistance (moderate vs. severe), and use of concomitant psychotropic medications (dichotomized as yes/no).

### Example 4

### Results

Depression severity—Infusion phase. Consistent with prior work, ketamine rapidly reduced MADRS total depression scores at 24-hours post-infusion (group by time interaction: standardized 3=−1.30, 95% CI: −1.89, −0.70; t=−4.29, df=150, p<0.0001; FIG. 9). This moderate-to-large linear effect corresponded to a 52% responder and 28% remitter rate in ketamine-treated participants and a 25% responder and 4% remitter rate in saline-treated participants [number-needed-to-treat (NNT) for response=3.7; NNT for remission=4.2].

Depression severity—NT phase (ASAT phase). In intent-to-treat linear mixed models, depression scores in the ketamine+ASAT group remained stably low over the 30-day acute phase relative to those in the saline+ASAT group (β=−0.61, 95% CI=−0.95, −0.28; t=−3.62, df=148, p=0.0004; FIG. 9), with no corresponding group-by-time interaction (β=0.009, 95% CI=−0.004, 0.021; t=1.39, df=568, p=0.164; FIG. 9), suggesting durability of effect over the 30-day window. By contrast, depression scores in the ketamine+sham group followed an increasing linear trajectory from 24 hours to 30 days, as the postketamine effect gradually waned and depression score levels approached those of the saline1ASAT group (group-by-time interaction relative to the saline+ASAT group: β=0.015, 95% CI=0.003, 0.03; t=2.35, df=568, p=0.019). While the above intent-to-treat statistical tests of linear trajectories over the full 30-day period were used for statistical hypothesis tests, pairwise post hoc contrasts at day 30 were used to generate effect size point estimates for further description of clinical impact at this specific time point. These effect size point estimates suggested a small effect favoring ketamine+ASAT relative to both saline+ASAT (β=−0.38, 95% CI=−0.78, 0.027) and ketamine+sham ((=−0.31, 95% CI=−0.75, 0.13).

All findings above were robustly upheld in sensitivity analyses controlling for covariates (see Examples 6-8).

Adequacy of blinding. As shown in Table S1 (FIG. 10), both patients and raters were functionally unblinded to infusion condition (ketamine vs. saline). While 35% of saline-treated patients incorrectly guessed that they received ketamine, 99% of ketamine-treated patients (all but one) correctly guessed that they received ketamine. In both groups (ketamine and saline-treated participants), clinician ratings of allocation were significantly correlated with degree of symptom reduction (% change from baseline MADRS) at the timepoint when blinding was assessed (infusion+30 days; r's≥0.46; p's≤0.001). By contrast, patient ratings of allocation were significantly correlated with degree of symptom reduction in the saline arm (r=0.49; p=0.001), but not the ketamine arm (r=0.01; p=0.95). These patterns suggest that, whereas clinician's correct guesses as to allocation may have been more uniformly driven by degree of clinical improvement, patient's first-hand knowledge of other infusion day experiences (e.g., dissociative side effects) likely contributed to their correct guesses (irrespective of clinical improvement).

In contrast, both patients and raters were adequately blinded to NT condition. Patients' mean response with respect to both conditions (active=54%; sham=49%; t₁₄₃=1.28; p=0.20) reflected the “I don't know” mid-point portion of the 0-100% response choice scale, as did the clinicians' responses (active=55%; sham=53%; t₁₄₇=0.60; p=0.55).

### Example 5

### DISCUSSION

In the present study, Cognitive Training—a low-cost, fully automated, non-invasive, brief (≤20 min/session, delivered over 8 sessions), computer-based intervention—was successful at extending the rapid antidepressant effect of a single ketamine infusion for at least 30 days. As in prior work, intravenous ketamine exerted a rapid antidepressant effect among treatment-resistant depressed patients. Ketamine infusion, relative to saline infusion, appeared also to successfully open a clinical window of opportunity, facilitating the efficient uptake of new positive self-representations to protect against the return of depression over the subsequent month (FIG. 9). By contrast, identical NT procedures, when given in the absence of pre-treatment with ketamine, exhibited no appreciable beneficial impact on depressive symptoms, over and above the non-specific effects of a saline infusion and the overarching clinical research context (e.g., repeated depression assessments). A second comparator group, ketamine followed by sham NT, followed the expected trajectory when a single ketamine infusion has been given as a stand-alone treatment, i.e., rapid decrease followed by gradual return of symptoms over the ensuing weeks. This represents the first study to test a synergistic pairing of ketamine with a behavioral intervention to include both a no-ketamine and a no-behavioral intervention control. Likewise, clinical trials pairing a wide range of posited “psychoplastogens” or other brain-based targeted approaches (e.g., neuromodulation) with behavioral treatments have almost universally neglected one or the other of these two critical comparators [Wilkinson S T, Holtzheimer P E, Gao S, Kirwin D S, Price R B: Leveraging Neuroplasticity to Enhance Adaptive Learning: The Potential for Synergistic Somatic-Behavioral Treatment Combinations to Improve Clinical Outcomes in Depression. 2019; 85(6):454-65]. The treatment protocol design described herein affords critical, new evidence for posited synergy between ketamine's neuroplasticity-enhancing acute impact and the introduction of protective learning delivered through behavioral treatment.

While this disclosure contemplates that a range of traditional and non-traditional behavioral treatments might work synergistically with rapid pharmacology, this study focused specifically on a NT approach, for both practical and scientific reasons. Practically, NT interventions are mechanistic and portable, and designed to be efficient, fitting well within the brevity (i.e., about 1 week) of ketamine's window-of-opportunity. By contrast, a full course of either traditional or computer-based cognitive-behavioral therapy (CBT) typically requires 12-16 1-hour sessions delivered weekly, and meta-analyses suggest effects of computer-based CBT are strongest only when supported by therapist coaching [Andersson G, Cuijpers P: Internet-based and other computerized psychological treatments for adult depression: a meta-analysis. Cogn Behav Ther. 2009; 38(4):196-205]. Scientifically, a robust evidence base suggests that implicit and explicit forms of cognition are distinct entities, and that implicit cognition (e.g., implicit self-concept) could be a more robust predictor of future behavior than explicit thought content [Greenwald A G, Poehlman T A, Uhlmann E L, Banaji M R: Understanding and using the Implicit Association Test: III. Meta-analysis of predictive validity. J Pers Soc Psychol. 2009; 97(1):17-41]. NT is designed to efficiently and directly target implicit cognition, yielding an innovative approach with the potential to efficiently yet profoundly shape future behavior and experience. Furthermore, by targeting a unitary and well-defined implicit cognitive mechanism, NT limits the influence of heterogeneous and non-specific factors that are likely influential in many traditional behavioral interventions, and for which the learning that occurs within a brief window may be more difficult to predict and constrain (e.g., positive vs. negative therapy session experiences).

NT specifically targeting implicit self-representations was selected based on prior work that suggested optimal synergy might be achieved when targeting this particular form of information processing after first priming brain plasticity with ketamine. Evidence for rapid plasticity in implicit self-representations following ketamine was found in two previous studies [Price R B, losifescu DV, Murrough J W, Chang L C, AI Jurdi R K, Iqbal S Z, Soleimani L, Charney D S, Foulkes A L, Mathew S J: Effects of ketamine on explicit and implicit suicidal cognition: a randomized controlled trial in treatment-resistant depression. Depress Anxiety. 2014; 31(4):335-43; Price R B, Nock M K, Charney D S, Mathew S J: Effects of intravenous ketamine on explicit and implicit measures of suicidality in treatment-resistant depression. Biol Psychiatry. 2009; 66(5):522-6], suggesting ketamine creates the requisite malleability in information processing within this domain. Similarly, prior neuroimaging work implicates shifts in prefrontal and striatal network activity and connectivity following ketamine (e.g., increased activation in the caudate and increased striatal-mPFC connectivity during face processing [Murrough J W, Collins K A, Fields J, DeWilde K E, Phillips M L, Mathew S J, Wong E, Tang C Y, Charney D S, losifescu DV: Regulation of neural responses to emotion perception by ketamine in individuals with treatment-resistant major depressive disorder. Transl Psychiatry. 2015; 5:e509]). As striatum and mPFC are widely implicated in appetitive conditioning in humans and rodents [Martin-Soelch C, Linthicum J, Ernst M: Appetitive conditioning: neural bases and implications for psychopathology. Neurosci Biobehav Rev. 2007; 31(3):426-40], these findings are also potentially consistent with a synergistic match between ketamine's neural effects and an appetitive conditioning paradigm, as was used here. Broadly, evaluative conditioning techniques have a large and robust literature in healthy controls [Hofmann W, De Houwer J, Perugini M, Baeyens F, Crombez G: Evaluative conditioning in humans: a meta-analysis. Psychol Bull. 2010; 136(3):390-421]; and in clinical samples, have previously shown strong, replicated acute clinical effects in an at-home (smartphone) delivery modality [Franklin J C, Fox K R, Franklin C R, Kleiman E M, Ribeiro J D, Jaroszewski A C, Hooley J M, Nock M K: A brief mobile app reduces nonsuicidal and suicidal self-injury: Evidence from three randomized controlled trials. J Consult Clin Psychol. 2016; 84(6):544-57]. The ability to both achieve and maintain gains via portable, highly dissemination-ready techniques is a crucial criterion for overcoming intractable barriers to access to care, particularly for underserved communities [Atdjian S, Vega W A: Disparities in mental health treatment in U.S. racial and ethnic minority groups: implications for psychiatrists. Psychiatr Serv. 2005; 56(12):1600-2]. Finally, the role of “depressive self-schemas” in creating vulnerability to negative affect is among the most long-standing and well-documented cognitive mechanisms mediating depression⁵³, suggesting strong clinical relevance for this mechanistic target.

Conclusions. The global burden of depression is extremely high, and is expected to continue to increase within the current context of a significant pandemic. Novel treatment approaches, particularly those that can provide efficient relief, are urgently needed. The treatment protocol described herein helps advance an integrative plasticity-based model of depression, revealing a novel synergistic treatment that urgently brings relief, and efficiently extends this relief via safe, low-cost, portable techniques. Alongside fully automated NT, ketamine itself has properties that could make it more dissemination-ready than many alternatives, including a favorable safety profile for isolated infusions, current FDA approval and wide, international medical use as a general anesthetic, and high ‘name recognition’ among patients and clinicians.

### Example 6

### Neurocognitive Training Technical Details

All Neurocognitive Training (active or sham) procedures were delivered in a private office on a laptop computer running E-Prime 2 software (Psychology Software Tools; Pittsburgh PA) and a Windows operating system. Positive and neutral word lists and photographs were comprised from previously validated stimulus sets used in prior research.

Each NT session was comprised of three consecutive blocks: (1) a subliminal lexical evaluative conditioning block; (2) a supraliminal lexical evaluative condition block; and (3) a pictorial evaluative conditioning block.

In the subliminal lexical evaluative conditioning block, 60 trials were presented consecutively in randomized order. Each trial consisted of a fixation string in the center of the screen (‘XXXX’; 500 ms), followed by a single letter probe (active NT: ‘I’, implicitly denoting ‘self’; sham NT: ‘A’) presented for 17 ms; followed by a positive word (active NT) or a neutral word (sham NT) presented for 17 ms; followed by a final probe string comprised of random letters, presented until a response was made. The participant's task was to press a key to indicate whether the final random letter string began with a vowel (left key; 50% of trials) or a consonant (right key; 50% of trials).

In the supraliminal lexical evaluative conditioning block, 60 trials were presented consecutively in randomized order. Each trial consisted of a fixation string (‘XXXX’; 500 ms), followed by a single letter probe (active NT: either ‘X’ or ‘I’; sham NT: either ‘X’ or ‘A’) presented for 500 ms; followed by either a real word or a random letter string, presented until a response was made. In the active NT group, the letter ‘X’ always preceded random letter strings and the letter ‘I’ (implicitly denoting ‘self’) always preceded positive words. In the sham NT group, the letter ‘X’ always preceded random letter strings and the letter ‘I’ always preceded negative words. The participant's task was to press a key to indicate whether the final letter string was a word (left key; 50% of trials) or a non-word (right key; 50% of trials).

In the pictorial evaluative conditioning block, 480 trials were presented consecutively in randomized order (with one self-paced break at the midpoint). Each trial consisted of a screen divided into 4 quadrants, and a single photographic probe presented until the participant made a mouse click in the correct quadrant; after mouse click, the first photograph was immediately replaced by a second photograph in the same location, which was presented for 400 ms. This trial is shown in FIG. 3. In the active NT condition, the first (probe) pictures on 33% of the trials consisted of one of three digital photographs taken of the participant at the screening visit, who were coached to use a neutral facial expression. Head-shot photographs were taken at a standardized distance, filling the full vertical frame, at three standardized angles (head-on, 450 left, 45° right). Whenever the first picture was a participant's self-photo, the second, replacement photo condition consisted of a happy facial expression, randomly selected from the happy expressions of seven male and seven female actors in the standardized NimStim image set [Tottenham N, Tanaka J W, Leon A C, McCarry T, Nurse M, Hare T A, Marcus D J, Westerlund A, Casey B J, Nelson C: The NimStim set of facial expressions: judgments from untrained research participants. Psychiatry Res 2009; 168(3):242-9]. On the remaining 66% of trials in the active NT condition, the first (probe) picture consisted of digital photographs of strangers in the same three orientations, taken from the neutral expression images in the standardized Karolinska Directed Emotional Faces (KDEF) image set (Lundqvist, D., Flykt, A., & Ohman, A.; 1998); and the second (replacement) photo consisted of either a second neutral facial expression (33% of trials) or an angry facial expression (33% of trials), randomly selected from the standardized angry and neutral expressions of seven male and seven female actors in the standardized NimStim image set. In the sham NT condition, the first (probe) pictures were all comprised of neutral expression images in the standardized Karolinska Directed Emotional Faces (KDEF) image set (Lundqvist, D., Flykt, A., & Ohman, A.; 1998). The second, replacement photos consisted of either a second neutral facial expression (66% of trials) or an angry facial expression (33% of trials), randomly selected from the standardized angry and neutral expressions of seven male and seven female actors in the standardized NimStim image set.

### Example 7

### Measurement of Covariates

At the screening assessment, participants completed general self-report demographic and medical history forms and supplied a full list of their current medications (including dose and indication). This information was used to quantify the following indices for use as covariates in sensitivity analyses: age, biological sex assigned at birth (as listed on one's birth certificate), and concurrent use of any concomitant psychotropic medication. Patients were queried on the date of their last psychotropic medication change to ensure >4 weeks between the last change and the screening assessments (which equated to roughly 6 weeks stable dose prior to infusion date), and were asked to maintain stable doses of medications and other psychiatric therapies (e.g., psychotherapy) until the infusion day and then throughout the 30-Day post-infusion acute phase study period. Severity of past treatment resistance to FDA-approved antidepressant medications, assessed by the clinical interviewer using the ATRQ and dichotomized as moderate (<3 failed trials in the current episode) or severe (≥3 failed trials in the current episode), was used to stratify randomization (in addition to sex assignment at birth) and as an additional covariate in sensitivity analyses.

### Example 8

### Adequacy of Blinding

At the final (+30 days) visit, adequacy of both patient and clinical rater blinding, for both infusion and NT allocation, was assessed using a 0-100 sliding scale (0=definitely placebo/sham; 50=1 don't know; 100=definitely ketamine/active). As shown in FIG. 10, Table S1, both patients and the rater were functionally unblinded to infusion condition (ketamine vs. saline). While 35% of saline-treated patients incorrectly guessed that they received ketamine, 99% of ketamine-treated patients (all but one) correctly guessed that they received ketamine. In both groups (ketamine and saline-treated participants), clinician ratings of allocation were significantly correlated with degree of symptom reduction (% change from baseline MADRS) at the timepoint when blinding was assessed (infusion+30 days; r's_0.46; p's<0.001). By contrast, patient ratings of allocation were significantly correlated with degree of symptom reduction in the saline arm (r=0.49; p=0.001), but not the ketamine arm (r=0.01; p=0.95). These patterns suggest that, whereas clinician's correct guesses as to allocation may have been more uniformly driven by degree of clinical improvement, patient's first-hand knowledge of other infusion day experiences (e.g., dissociative side effects) likely contributed to their correct guesses (irrespective of clinical improvement).

In contrast, both patients and the rater were adequately blinded to NT condition (FIG. 10, Table S1). Patients' mean response with respect to both conditions (active=54%; sham=49%; t₁₄₃=1.28; p=0.20) reflected the “I don't know” mid-point portion of the 0-100% response choice scale, as did the clinicians' responses (active=55%; sham=53%; t₁₄₇=0.60; p=0.55).

Though very few prior ketamine studies have reported explicit measurements of blinding success, the prospect of inadequate blinding has been noted consistently as a challenge when conducting RCTs of ketamine given its unique, acute impact on cognition-even when utilizing a psychoactive comparison arm (e.g., midazolam) [Wilkinson S T, Farmer C, Ballard E D, Mathew S J, Grunebaum M F, Murrough J W, Sos P, Wang G, Gueorguieva R, Zarate C A, Jr.: Impact of midazolam vs. saline on effect size estimates in controlled trials of ketamine as a rapid-acting antidepressant. Neuropsychopharmacology 2019; 44(7):1233-8; Murrough J W, Iosifescu D V, Chang L C, A I Jurdi R K, Green C M, Perez A M, Iqbal S, Pillemer S, Foulkes A, Shah A, Charney D S, Mathew S J: Antidepressant efficacy of ketamine in treatment-resistant major depression: A Two-site randomized controlled trial. Am J Psychiatry 2013; 170(10):1134-42]. Here, an inert placebo was selected because a large body of prior work already substantiated ketamine's clinical efficacy in depression, relative to both inert and psychoactive placebo conditions) [[Wilkinson S T, Farmer C, Ballard E D, Mathew S J, Grunebaum M F, Murrough J W, Sos P, Wang G, Gueorguieva R, Zarate C A, Jr.: Impact of midazolam vs. saline on effect size estimates in controlled trials of ketamine as a rapid-acting antidepressant. Neuropsychopharmacology 2019; 44(7):1233-8; Xu Y, Hackett M, Carter G, Loo C, Galvez V, Glozier N, Glue P, Lapidus K, McGirr A, Somogyi A A, Mitchell P B, Rodgers A: Effects of low-dose and very low-dose dose ketamine among patients with major depression: a systematic review and meta-analysis. Int J Neuropsychopharmacol 2015; Newport D J, Carpenter L L, McDonald W M, Potash J B, Tohen M, Nemeroff C B, Treatments APACoRTFoNBa: Ketamine and Other NMDA Antagonists: Early Clinical Trials and Possible Mechanisms in Depression. Am J Psychiatry 2015; 172(10):950-66; Caddy C, Amit B H, McCloud T L, Rendell J M, Furukawa T A, McShane R, Hawton K, Cipriani A: Ketamine and other glutamate receptor modulators for depression in adults. Cochrane Database Syst Rev 2015; September 23(9):CD011612], and the present design facilitated clean comparisons of ketamine's potential synergy with NT, without introducing the potential ambiguity or confounding (either positive or detrimental) neurocognitive effects of a second psychoactive substance. The fact that a significant response to saline (25% responders at 24-hours; reductions from baseline maintained throughout the 30-day assessment period—FIG. 9) was observed in our sample provides some reassurance that the saline arm successfully controlled for many non-specific factors (including, but not limited to, expectancy effects) that influence outcomes in depression treatment trials [Dunlop B W, Thase M E, Wun C C, Fayyad R, Guico-Pabia C J, Musgnung J, Ninan P T: A meta-analysis of factors impacting detection of antidepressant efficacy in clinical trials: the importance of academic sites. Neuropsychopharmacology 2012; 37(13):2830-6].

### Example 9

Described below is an analysis of self-report data, collected during a 1-year naturalistic follow-up, to explore the full extent of the durability of the automated neurocognitive training protocol described in the examples herein.

154 adult patients with treatment-resistant depression, on stable psychiatric treatments, were randomized to one of three treatment conditions [Price R B, Spotts C, Panny B, Griffo A, Degutis M, Cruz N, et al.: A Novel, Brief, Fully Automated Intervention to Extend the Antidepressant Effect of a Single Ketamine Infusion: A Randomized Clinical Trial. Am J Psychiatry. 2022:appiajp20220216]: a single infusion of ketamine (0.5 mg/kg) followed by 4 days (30-40 min/day) of ASAT, a digital therapeutic designed to leverage a hypothesized “window” of plasticity following ketamine to target implicit thought patterns related to oneself; ketamine followed by sham ASAT (identical computer exercises lacking therapeutic and self-relevant content); or saline infusion followed by active ASAT. Throughout a 1-year naturalistic follow-up period, patients completed the Quick Inventory for Depressive Symptoms (QIDS-SR [Rush A J, Trivedi M H, Ibrahim H M, Carmody T J, Arnow B, Klein D N, et al.: The 16-Item Quick Inventory of Depressive Symptomatology (QIDS), Clinician Rating (QIDS-C) and Self-Report (QIDS-SR): A Psychometric evaluation in patients with chronic major depression. Biol Psychiatry. 2003; 54:573-83]), designated as the primary outcome during the follow-up phase. Surveys were solicited at the following timepoints relative to the infusion date: +30, +60, +90, +120, +150, +180 and +360 days.

Timepoint and Group (using saline+ASAT as the reference group) were included as categorical factors in an intent-to-treat, hierarchical linear model (which automatically estimates missing values) predicting QIDS-SR total scores, covarying pre-infusion baseline QIDS-SR scores.

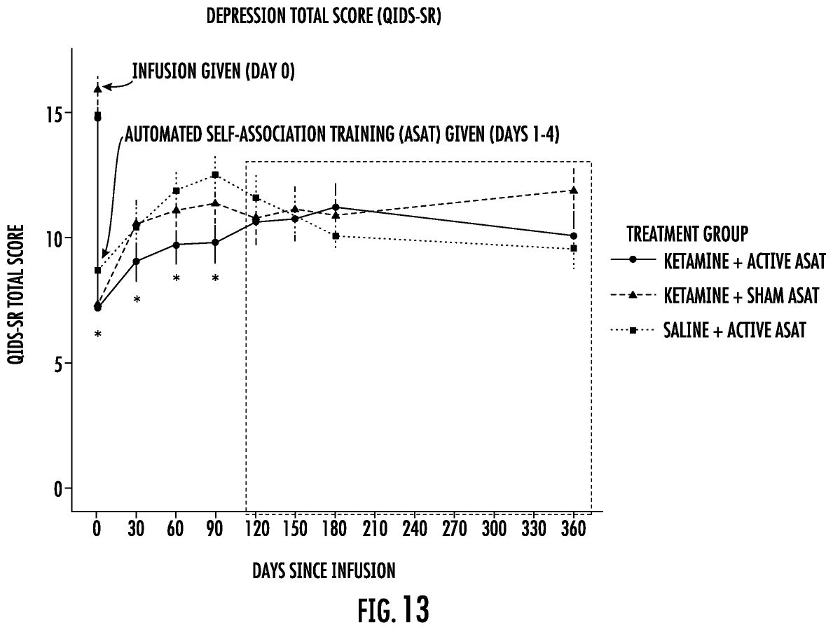

A significant Timepoint*Group interaction was observed in the full model (standardized F(12,718)=3.15; p=0.0002). To dissect this interaction, given lack of a priori knowledge regarding potential durability of this first-of-its-kind intervention approach, visual inspection of symptom trajectories was used to identify a data-driven breakpoint at which error bars became overlapping, visible at ≥120 days post-infusion (FIG. 13).

In FIG. 13, the blue line represents ketamine+active Automated Self-Association Training (ASAT), n=53; the orange line represents ketamine+sham ASAT, n=50; and the red line represents saline+active ASAT, n=51. Line plots represent mean values and error bars represent standard error of the mean within each timepoint and treatment group. Grey shading indicates “late” Timeframe where no significant pairwise group differences were observed at any timepoint, between any two groups. Pre-infusion baseline and 1-day post-infusion (immediate post-ketamine or saline, prior to the onset of active/sham ASAT) are depicted here for comprehensive visualization, but follow-up period statistical analyses focused on the period from infusion+30 days forward (as described in the text). * indicates timepoint with significant decrease in Ketamine+Active ASAT arm relative to Saline+Active ASAT arm per unpaired t-tests.

To minimize multiple comparisons, pairwise planned contrasts were then used to probe group means during these data-driven “early” (≤90 days) and “late” (≥120 days) timeframes. As illustrated in FIG. 13, in the “early” Timeframe, there was a significant effect of group for ketamine+ASAT vs. saline+ASAT (R[95% CI]=−0.37[−0.71, −0.04]; SE=0.168; t(144)=−2.21; p=0.0287), while there was no corresponding significant effect when ketamine was given without ASAT (ketamine+sham) compared to saline+ASAT (β=−0.197[−0.54, 0.14]; SE=0.172; t(144)=−1.147; p=0.253). By contrast, in the “late” Timeframe, there were no longer any significant differences between groups (ket+ASATvs. saline+ASAT: β=0.179; SE=0.168; t(144)=1.068; p=0.287; ket+sham vs. saline+ASAT: β=0.111; SE=0.170; t(144)=0.653; p=0.515).

This study is the first to demonstrate that the rapid effects of ketamine can be made more enduring with simple, portable, digital techniques that would be relatively easy to provide to patients in a wide range of settings. One 40-minute drug administration, followed by 4 days (30-40 min/day) of digital exercises, created a statistical effect on depression that endured for 3 months according to the new follow-up phase data presented here-even in the context of naturalistic treatment changes across all arms.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A method for treating a patient having a mental disorder comprising:
administering a drug having an antidepressant effect to the patient; and
administering a neurocognitive training protocol to the patient during a critical time period following administration of the drug, wherein administering the neurocognitive training protocol to the patient comprises delivering, using a computing device, a plurality of automated neurocognitive training sessions to the patient, wherein a plurality of conditioning sequences are presented to the patient during delivery of each of the automated neurocognitive training sessions, each respective conditioning sequence comprising a respective non-self-relevant stimulus and a respective self-relevant stimulus.

2. The method of claim 1, wherein:
a first automated neurocognitive training session and a second automated neurocognitive training session are delivered to the patient on a first day, and
a third automated neurocognitive training session and a fourth automated neurocognitive training session are delivered to the patient on a second day.

3. The method of claim 2, wherein a period of time between delivery of the first and second automated neurocognitive training sessions to the patient is about 20 minutes or more.

4. The method of claim 2, wherein a period of time between delivery of the third and fourth automated neurocognitive training sessions to the patient is about 20 minutes or more.

5. The method of claim 1, wherein each of the automated neurocognitive training sessions has a length between about 15 minutes and about 20 minutes.

6. The method of claim 1, wherein the plurality of automated neurocognitive training sessions is between about 2 sessions and about 14 sessions.

7. The method of claim 1, wherein the plurality of automated neurocognitive training sessions is about 8 sessions.

8. The method of claim 1, wherein each respective conditioning sequence further comprises a task, the task being presented to the patient following or in relation to presentation of a respective non-self-relevant and self-relevant stimuli pair.

9. The method of claim 1, wherein the respective non-self-relevant stimulus and/or the respective self-relevant stimulus for a respective conditioning sequence is an auditory stimulus.

10. The method of claim 1, wherein the respective non-self-relevant stimulus and/or the respective self-relevant stimulus for a respective conditioning sequence is a visual stimulus.

11. The method of claim 1, wherein the respective self-relevant stimulus for a respective conditioning sequence comprises a sound, image, or word that is associated with the patient.

12. The method of claim 11, wherein the respective non-self-relevant stimulus for the respective conditioning sequence comprises a sound, image, or word that is associated with a positive meaning.

13. The method of claim 11, wherein the respective non-self-relevant stimulus for the respective conditioning sequence comprises a sound, image, or word that is associated with a life-affirming meaning.

14. The method of claim 1, wherein the respective non-self-relevant stimulus and the respective self-relevant stimulus for a respective conditioning sequence are included in an n×m matrix comprising a plurality of visual stimuli.

15. The method of claim 14, wherein administering the neurocognitive training protocol to the patient further comprises receiving, from the patient, a selection matching the respective non-self-relevant stimulus and the respective self-relevant stimulus for the respective conditioning sequence.

16. The method of claim 1, wherein one or more of the plurality of conditioning sequences comprises a subliminal presentation of a non-self-relevant stimulus or a self-relevant stimulus.

17. The method of claim 1, wherein one or more of the plurality of conditioning sequences comprises a supraliminal presentation of a non-self-relevant stimulus or a self-relevant stimulus.

18. The method of claim 1, wherein the computing device comprises a processor and a memory operably coupled to the processor, the memory having computer-executable instructions stored thereon.

19. The method of claim 18, wherein the memory has computer-executable instructions stored thereon, that when executed by the processor, cause the processor to generate each respective conditioning sequence.

20. The method of claim 19, wherein the computing device further comprises a display device operably coupled to the processor, and wherein the memory has computer-executable instructions stored thereon, that when executed by the processor, cause the processor to:
generate graphical data for each respective conditioning sequence; and
cause the graphical data to be displayed on the display device.

21. The method claim 1, wherein the critical time period is a period during which the drug acutely proliferates synaptic contacts within the patient's brain.

22. The method of claim 21, wherein the critical time period is between about 2 hours and about 14 days following administration of the drug.

23. The method of claim 21, wherein the critical time period is between about 1 day and about 5 days following administration of the drug.

24. The method of claim 1, wherein the drug is a rapid-acting antidepressant.

25. The method of claim 24, wherein the rapid-acting antidepressant is ketamine.

26. The method of claim 1, wherein the drug is brexanolone, zuranolone, rapastinel, scopolamine, or a psychedelic.

27. The method of claim 1, wherein the mental disorder is depression.

28. The method of claim 1, wherein the mental disorder is suicidal behavior.

29. A computer-implemented method for administering a neurocognitive training protocol to a patient having a mental disorder, the patient having received a drug having an antidepressant effect, and the neurocognitive training protocol being administered during a critical time period following administration of the drug to the patient, the computer-implemented method comprising:
delivering a plurality of automated neurocognitive training session to the patient, wherein a plurality of conditioning sequences are presented to the patient during delivery of each of the automated neurocognitive training sessions, each respective conditioning sequence comprising a respective non-self-relevant stimulus and a respective self-relevant stimulus.

30. The computer-implemented method of claim 29, wherein:
a first automated neurocognitive training session and a second automated neurocognitive training session are delivered to the patient on a first day, and
a third automated neurocognitive training session and a fourth automated neurocognitive training session are delivered to the patient on a second day.

31. The computer-implemented method of claim 30, wherein a period of time between delivery of the first and second automated neurocognitive training sessions to the patient is about 20 minutes or more.

32. The computer-implemented method of claim 30, wherein a period of time between delivery of the third and fourth automated neurocognitive training sessions to the patient is about 20 minutes or more.

33. The computer-implemented method of claim 29, wherein each of the automated neurocognitive training sessions has a length between about 15 minutes and about 20 minutes.

34. The computer-implemented method of claim 29, wherein the plurality of automated neurocognitive training sessions is between about 2 sessions and about 14 sessions.

35. The computer-implemented method of claim 29, wherein the plurality of automated neurocognitive training sessions is about 8 sessions.

36. The computer-implemented method of claim 29, wherein each respective conditioning sequence further comprises a task, the task being presented to the patient following or in relation to presentation of a respective non-self-relevant and self-relevant stimuli pair.

37. The computer-implemented method of claim 29, wherein the respective non-self-relevant stimulus and/or the respective self-relevant stimulus for a respective conditioning sequence is an auditory stimulus.

38. The computer-implemented method of claim 29, wherein the respective non-self-relevant stimulus and/or the respective self-relevant stimulus for a respective conditioning sequence is a visual stimulus.

39. The computer-implemented method of claim 29, wherein the respective self-relevant stimulus for a respective conditioning sequence comprises a sound, image, or word that is associated with the patient.

40. The computer-implemented method of claim 39, wherein the respective non-self-relevant stimulus for the respective conditioning sequence comprises a sound, image, or word that is associated with a positive meaning.

41. The computer-implemented method of claim 39, wherein the respective non-self-relevant stimulus for the respective conditioning sequence comprises a sound, image, or word that is associated with a life-affirming meaning.

42. The computer-implemented method of claim 29, wherein the respective non-self-relevant stimulus and the respective self-relevant stimulus for a respective conditioning sequence are included in an n×m matrix comprising a plurality of visual stimuli.

43. The computer-implemented method of claim 42, further comprising receiving a selection matching the respective non-self-relevant stimulus and the respective self-relevant stimulus for the respective conditioning sequence.

44. The computer-implemented method of claim 29, wherein one or more of the plurality of conditioning sequences comprises a subliminal presentation of a non-self-relevant stimulus or a self-relevant stimulus.

45. The computer-implemented method of claim 29, wherein one or more of the plurality of conditioning sequences comprises a supraliminal presentation of a non-self-relevant stimulus or a self-relevant stimulus.

46. The computer-implemented method of claim 29, further comprising generating each respective conditioning sequence.

47. The computer-implemented method of claim 46, further comprising:
generating graphical data for each respective conditioning sequence; and
causing the graphical data to be displayed on a display device.

48. The computer-implemented method of claim 29, wherein the critical time period is a period during which the drug acutely proliferates synaptic contacts within the patient's brain.

49. The computer-implemented method of claim 48, wherein the critical time period is between about 2 hours and about 14 days following administration of the drug.

50. The computer-implemented method of claim 48, wherein the critical time period is between about 1 day and about 5 days following administration of the drug.

51. A system for administering a neurocognitive training protocol to a patient having a mental disorder, the patient having received a drug having an antidepressant effect, and the neurocognitive training protocol being administered during a critical time period following administration of the drug to the patient, the system comprising:
a processor and a memory operably coupled to the processor, the memory having computer-executable instructions stored thereon that, when executed by the processor, cause the processor to:
deliver a plurality of automated neurocognitive training session to the patient, wherein a plurality of conditioning sequences are presented to the patient during delivery of each of the automated neurocognitive training sessions, each respective conditioning sequence comprising a respective non-self-relevant stimulus and a respective self-relevant stimulus.

52. The system of claim 51, wherein:
a first automated neurocognitive training session and a second automated neurocognitive training session are delivered to the patient on a first day, and
a third automated neurocognitive training session and a fourth automated neurocognitive training session are delivered to the patient on a second day.

53. The system of claim 52, wherein a period of time between delivery of the first and second automated neurocognitive training sessions to the patient is about 20 minutes or more.

54. The system of claim 52, wherein a period of time between delivery of the third and fourth automated neurocognitive training sessions to the patient is about 20 minutes or more.

55. The system of claim 51, wherein each of the automated neurocognitive training sessions has a length between about 15 minutes and about 20 minutes.

56. The system of claim 55, wherein the plurality of automated neurocognitive training sessions is between about 2 sessions and about 14 sessions.

57. The system of claim 55, wherein the plurality of automated neurocognitive training sessions is about 8 sessions.

58. The system of claim 51, wherein each respective conditioning sequence further comprises a task, the task being presented to the patient following or in relation to presentation of a respective non-self-relevant and self-relevant stimuli pair.

59. The system of claim 51, wherein the respective non-self-relevant stimulus and/or the respective self-relevant stimulus for a respective conditioning sequence is an auditory stimulus.

60. The system of claim 51, wherein the respective non-self-relevant stimulus and/or the respective self-relevant stimulus for a respective conditioning sequence is a visual stimulus.

61. The system of claim 51, wherein the respective self-relevant stimulus for a respective conditioning sequence comprises a sound, image, or word that is associated with the patient.

62. The system of claim 61, wherein the respective non-self-relevant stimulus for the respective conditioning sequence comprises a sound, image, or word that is associated with a positive meaning.

63. The system of claim 61, wherein the respective non-self-relevant stimulus for the respective conditioning sequence comprises a sound, image, or word that is associated with a life-affirming meaning.

64. The system of claim 51, wherein the respective non-self-relevant stimulus and the respective self-relevant stimulus for a respective conditioning sequence are included in an n×m matrix comprising a plurality of visual stimuli.

65. The system of claim 51, wherein one or more of the plurality of conditioning sequences comprises a subliminal presentation of a non-self-relevant stimulus or a self-relevant stimulus.

66. The system of claim 51, wherein one or more of the plurality of conditioning sequences comprises a supraliminal presentation of a non-self-relevant stimulus or a self-relevant stimulus.

67. The system of claim 51, wherein the memory has further computer-executable instructions stored thereon that, when executed by the processor, cause the processor to generate each respective conditioning sequence.

68. The system of claim 67, wherein the memory has further computer-executable instructions stored thereon that, when executed by the processor, cause the processor to:
generate graphical data for each respective conditioning sequence; and
cause the graphical data to be displayed on a display device.

69. A method for treating a patient having a mental disorder comprising:
administering a drug having an antidepressant effect to the patient; and
administering a neurocognitive training protocol to the patient during a critical time period following administration of the drug, wherein administering the neurocognitive training protocol to the patient comprises delivering, using a computing device, a plurality of automated neurocognitive training sessions to the patient, wherein each respective automated neurocognitive training session comprises:
presenting, using the computing device, an n×m matrix comprising a plurality of visual stimuli to the patient, the plurality of visual stimuli comprising a first matching pair of non-self-relevant and self-relevant visual stimuli; and
receiving, using the computing device, a selection by the patient of the first matching pair of non-self-relevant and self-relevant visual stimuli.

70. The method of claim 69, wherein each respective automated neurocognitive training session further comprises:
presenting, using the computing device, a second matching pair of non-self-relevant and self-relevant visual stimuli to the patient; and
presenting, using the computing device, a task to the patient following or in relation to presentation of the second matching pair of non-self-relevant and self-relevant visual stimuli.

71. The method of claim 70, wherein each respective automated neurocognitive training session further comprises:
presenting, using the computing device, a self-relevant visual stimulus to the patient;
presenting, using the computing device, a task to the patient following or in relation to presentation of the self-relevant stimulus; and
presenting, using the computing device, a positive non-self-relevant visual stimulus following successful completion of the task by the patient.

72. The method of claim 69, wherein each of the automated neurocognitive training sessions has a length between about 15 minutes and about 20 minutes.

73. The method of claim 69, wherein the critical time period is a period during which the drug acutely proliferates synaptic contacts within the patient's brain.

74. The method of claim 73, wherein the critical time period is between about 2 hours and about 14 days following administration of the drug.

75. The method of claim 73, wherein the critical time period is between about 1 day and about 5 days following administration of the drug.

76. A computer-implemented method for administering a neurocognitive training protocol to a patient having a mental disorder, the patient having received a drug having an antidepressant effect, and the neurocognitive training protocol being administered during a critical time period following administration of the drug to the patient, the computer-implemented method comprising:
presenting an n×m matrix comprising a plurality of visual stimuli to the patient, the plurality of visual stimuli comprising a first matching pair of non-self-relevant and self-relevant visual stimuli; and
receiving a selection by the patient of the first matching pair of non-self-relevant and self-relevant visual stimuli.

77. A system for administering a neurocognitive training protocol to a patient having a mental disorder, the patient having received a drug having an antidepressant effect, and the neurocognitive training protocol being administered during a critical time period following administration of the drug to the patient, the system comprising:
a processor and a memory operably coupled to the processor, the memory having computer-executable instructions stored thereon that, when executed by the processor, cause the processor to:
present an n×m matrix comprising a plurality of visual stimuli to the patient, the plurality of visual stimuli comprising a first matching pair of non-self-relevant and self-relevant visual stimuli; and
receive a selection by the patient of the first matching pair of non-self-relevant and self-relevant visual stimuli.

78. A method for treating a patient having a mental disorder comprising:
administering a drug having an antidepressant effect to the patient; and
administering a neurocognitive training protocol to the patient during a critical time period following administration of the drug, wherein administering the neurocognitive training protocol to the patient comprises delivering, using a computing device, a plurality of automated neurocognitive training sessions to the patient, wherein a plurality of conditioning sequences are presented to the patient during delivery of each of the automated neurocognitive training sessions.

79. A method for conditioning a therapeutic association in a patient comprising:
administering a neuroplasticity-enhancing treatment to the patient; and
administering a neurocognitive training protocol to the patient during a critical time period following administration of the neuroplasticity-enhancing treatment, wherein administering the neurocognitive training protocol to the patient comprises delivering, using a computing device, one or more conditioning sequences configured to condition a therapeutic association to the patient.

80. The method of claim 79, wherein the neuroplasticity-enhancing treatment is a drug or a neuromodulatory intervention.
